SEQUENCE LISTING (3 sequences)

SEQ ID NO: 1 - DNA, 22 bp, Artificial Sequence
  Synthetic primer
agttgaggggactttcccaggc

SEQ ID NO: 2 - DNA, 21 bp, Artificial Sequence
  Synthetic primer
aatcggtgtctccaacttcaa

SEQ ID NO: 3 - DNA, 21 bp, Artificial Sequence
  Synthetic primer
aaaatctccctaaatcataca

(19) United States
(12) Patent Application Publication
(10) Pub. No.: US 2009/0270490 A1
(43) Pub. Date: Oct. 29, 2009

(54) METHODS INVOLVING ALDOSE REDUCTASE INHIBITION

(75) Inventors: Satish K. Srivastava, Galveston, TX (US); Kota V. Ramana, Galveston, TX (US)

Correspondence Address: FULBRIGHT & JAWORSKI L.L.P., 600 CONGRESS AVE., SUITE 2400, AUSTIN, TX, 78701, US

(73) Assignee: THE BOARD OF REGENTS OF THE UNIVERSITY OF TEXAS SYSTEM

(21) Appl. No.: 12/429,894

(22) Filed: Apr. 24, 2009

Related U.S. Application Data
(60) Provisional application No. 61/047,745, filed on Apr. 24, 2008.

(51) Int. Cl. A61K 31/7088; A61K 31/4164

(52) U.S. Cl. 514/44 R; 514/385

(57) ABSTRACT

Embodiments of the invention include methods and compositions involving aldose reductase inhibitors for the treatment of inflammation, including inflammatory bowel disease, macular degeneration, or posterior capsule opacification.

This application claims priority to U.S. Application No. 61/047,745 filed on Apr. 24, 2008, the entire disclosure of which are specifically incorporated herein by reference in its entirety

The invention was made with government support undergrants GM71036 and DK36118 awarded by the National Institute of Health. The government has certain rights in the invention.

### BACKGROUND OF THE INVENTION

I. Field of the Invention

Embodiments of this invention are related generally to physiology and medicine. More specifically, this invention is related to aldose reductase inhibitors (ARIs) and their use in treating and ameliorating inflammatory bowel disease (IBD), macular degeneration, and posterior capsule opacification.

II. Background

Aldose reductase (AR) catalyzes the reduction of a wide range of aldehydes (Bhatnager and Srivastava, 1992). The substrates of the enzyme range from aromatic and aliphatic aldehydes to aldoses such as glucose, galactose, and ribose. The reduction of glucose by AR is particularly significant during hyperglycemia and increased flux of glucose via AR has been etiologically linked to the development of secondary diabetic complications (Bhatnager and Srivastava, 1992; Yabe-Nishimura, 1998). However, recent studies showing that AR is an excellent catalyst for the reduction of lipid peroxidation-derived aldehydes and their glutathione conjugates (Srivastava et al., 1995; Vander Jagt et al., 1995; Srivastava et al., 1998; Srivastava et al., 1999; Dixit et al., 2000; Ramana et al., 2000) suggest that in contrast to its injurious role during diabetes, under normal glucose concentration, AR may be involved in protection against oxidative and electrophilic stress. The antioxidant role of AR is consistent with the observations that in a variety of cell types AR is upregulated by oxidants such as hydrogen peroxide (Spycher et al., 1997), lipid peroxidation-derived aldehydes (Ruef et al., 2000; Rittner et al., 1999), advanced glycosylation end products (Nakamura et al., 2000) and nitric oxide (Seo et al., 2000). The expression of the enzyme is also increased under several pathological conditions associated with increased oxidative or electrophilic stress such as iron overload (Barisani et al., 2000), alcoholic liver disease (O'Connor et al., 1999), heart failure (Yang et al., 2000), myocardial ischemia (Shinmura et al., 2000), vascular inflammation (Rittner et al., 1999) and restenosis (Ruef et al., 2000), and various forms of cancer.

Inhibitors of aldose reductase have been indicated for some conditions and diseases, such as diabetes complications, ischemic damage to non-cardiac tissue, Huntington's disease. See U.S. Pat. Nos. 6,696,407, 6,127,367, 6,380,200, which are all hereby incorporated by reference. In some cases, the role in which aldose reductase plays in mechanisms involved in the condition or disease are known. For example, in U.S. Pat. No. 6,696,407 indicates that an aldose reductase inhibitors increase striatal ciliary neurotrophic factor (CNTF), which has ramifications for the treatment of Huntington's Disease. In other cases, however, the way in which aldose reductase or aldose reductase inhibitors work with respect to a particular disease or condition are not known.

Therefore, the role of aldose reductase in a number of diseases and conditions requires elucidation, as patients with these diseases and conditions continue to require new treatments. Thus, there is a need for preventative and therapeutic methods involving aldose reductase and aldose reductase inhibitors.

### SUMMARY OF THE INVENTION

Embodiments of the invention are directed to methods of treating, preventing, or reducing symptoms related to IBD, macular degeneration, macular edema, AMD (age related macular degeneration), or posterior capsule opacification in a subject comprising administering to a subject diagnosed with or at risk of developing IBD, macular degeneration, or posterior capsule opacification an amount of a pharmaceutically acceptable composition comprising an aldose reductase inhibitor (ARI) sufficient to prevent, ameliorate, or attenuate IBD, macular degeneration, or posterior capsule opacification.

The composition may be administered 1, 2, 3, 4, 5, 6, or more times and may be administered over 1, 2, 3, 4, 5, 6, 7, or more minutes, hours, days or weeks. In certain aspects, the aldose reductase inhibitor is administered to the patient as a prodrug. Typically, prodrug is an inactive or less active form of a drug that is metabolized or converted in vivo to an active or more active form. ARI compositions can be administered directly, locally, topically, orally, ocularly, endoscopically, intratracheally, intravitreously, intrabronchially, intratumorally, intravenously, intralesionally, intramuscularly, intraperitoneally, regionally, percutaneously, or subcutaneously. In certain aspects the ARI is administered orally or by inhalation or instillation, e.g., by inhaler or other aersol delivery devices. In other aspects an aldose reductase inhibitor can be coupled or coated to or on a surface, e.g., surgical implants including replacement lenses.

In certain embodiments the aldose reductase inhibitor is a peptide, a peptide mimetic, a small molecule, or an inhibitory RNA. The aldose reductase inhibitor can be an siRNA or other inhibitory nucleic acid, a carboxylic acid, a hydantoins, a pyridazinone, or a pharmaceutically acceptable derivative thereof. In particular aspects the aldose reductase inhibitor is fidarestat, sorbinil, epalrestat, ponalrestat, methosorbinil, risarestat, imirestat, ALO-1567, quercetin, zopolrestat, AD-5467, NZ-314, M-16209, minalrestat, AS-3201, WP-921, luteolin, tolrestat, EBPC, fidarestat, or a pharmaceutically acceptable derivative thereof. In certain embodiments the aldose reductase inhibitor is fidarestat. In certain embodiments the aldose reductase inhibitor is not a nitric oxide inducer. The aldose reductase inhibitor can be administered at a dose of 0.01, 0.1, 1, 5, 10, 20, 40, 50, 100, 200, 400, 800, 1200 to 1500 ng/day, mg/day, ng/kg/day, or mg/kg/day, including all ranges and values there between.

Other embodiments of the invention are discussed throughout this application. Any embodiment discussed with respect to one aspect of the invention applies to other aspects of the invention as well and vice versa. The embodiments in the Example section are understood to be embodiments of the invention that are applicable to all aspects of the invention.

The terms “inhibiting,” “reducing,” or “prevention,” or any variation of these terms, when used in the claims and/or the specification includes any measurable decrease or complete inhibition to achieve a desired result.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed herein can be implemented with respect to any method or composition of the invention, and vice versa. Furthermore, compositions and kits of the invention can be used to achieve methods of the invention.

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1C. AR inhibition prevents the proliferation of lens epithelial cells in the pig eye capsular bag. Pig eye capsular bags were prepared by capsulorhexis and incubated in DMEM with 10% FBS and 1% penicillin/streptomycin with or without (FIG. 1A) sorbinil (50 μM) and (FIGS. 1B-C) fidarestat (10-30 μM) for 7 days in humidified incubator at 37° C. with 95% O₂ and 5% CO₂. The lens epithelial cells from capsular bags were harvested, washed, re-suspended in PBS and trypan blue positive cells were counted by using a hemocytometer. The bars represent Mean±SD (n=6). #P<0.03; ##P<0.001 Vs Control. Sorb, sorbinil; fida, fidarestat.

FIGS. 2A-2B. AR inhibition prevents the expression of differentiation markers in the lens epithelium of pig eye capsular bag. Pig eye capsular bags were prepared by capsulorhexis and cultured with or without AR inhibitor, (FIG. 2A) sorbinil (50 μM) and (FIG. 2B) fidarestat (30 μM) for 7 days. The lens epithelial cells were harvested from capsular bag, pooled together from each batch, washed and lysed in RIPA lysis buffer. Equal amounts of protein were subjected to Western blot analysis using specific antibodies against α-SMA, β-crystallin, and ICAM-1. The blots were striped and re-probed with antibodies against β-actin as loading control. Numbers below the blots show the fold change in the expression of proteins. A representative blot from 3 individual analyses is shown.

FIGS. 3A-3C. AR inhibition prevents b-FGF-induced proliferation of HLEC. Growth-arrested HLEC without or with AR inhibitors, sorbinil and zopolrestat (20 μM) were stimulated with (FIG. 3A) b-FGF (0, 10, 20, 50, and 1.00 ng/mL) or (FIGS. 3B-C) 20 ng/ml for 24 h. Cell viability was determined by MTT assay (FIG. 3A, 3C) or by cell counting (FIG. 3B). The bars represent mean±SD (n=4). ^{#}p<0.01 vs control; ^{# #p<}0.01 vs b-FGF treated; *p<0.05 vs b-FGF treated. Sorb (S), sorbinil; zopol (Z), zopolrestat; FGF (F), basic-fibroblast growth factor.

FIGS. 4A-4C. AR inhibition prevents the b-FGF-induced expression of differentiation markers in HLEC. (FIG. 4A) Growth-arrested HLEC without or with AR inhibitors (sorbinil and zopolrestat; 20 μM) and (FIG. 4B) untransfected (C), AR siRNA (ARsi) or control siRNA (Csi) transfected were stimulated with b-FGF (50 ng/ml) for 24 h. After incubation the cells were harvested, washed and lysed in RIPA lysis buffer. Equal amounts of protein (40 μg) were subjected to Western blot analysis using specific antibodies against α-SMA, β-crystallin, ICAM-1 and GAPDH. A representative blot from 3 individual analyses is shown. Sorb, sorbinil; zopol, zopolrestat; FGF, basic-fibroblast growth factor. (FIG. 4C) The cell lysate was used for TGF-β1 analysis by Western blot and the culture media from (FIG. 4A) was used for determination of TGF-β1 level by ELISA. Bars represent mean±SD (n=3). *p<0.05 vs Control.

FIGS. 5A-5B. AR inhibition attenuates the b-FGF-induced activation of NF-κB in HLEC. Growth-arrested HLEC without or with AR inhibitors (sorbinil and zopolrestat; 20 μM) were stimulated with b-FGF (50 ng/ml) for 1 h. (FIG. 5A) The equal amounts of nuclear proteins were subjected to EMSA, and (FIG. 5B) HLEC were transiently transfected with the pNF-κB-SEAP reporter vector. The cells treated without or with AR inhibitors (sorbinil and zopolrestat; 20 μM) were incubated with 50 ng/mL of b-FGF. After 48 hours, the culture supernatants were assayed for SEAP activity with a chemiluminescence kit according to the supplier's instructions. Bars represent Mean±SD (n=4). ^{#}p<0.001 Vs. Control and **p<0.01 vs b-FGF. Sorb, sorbinil; zopol, zopolrestat; FGF, basic-fibroblast growth factor.

FIG. 6. AR inhibition prevents b-FGF-induced activation of MAPK in HLEC. Growth-arrested HLEC without or with AR inhibitor, zopolrestat (20 μM), were treated with b-FGF (50 ng/ml) for indicated time periods. Equal amount of cytosolic extracts were subjected to Western blot analysis using antibodies against phospho- and total-ERK1/2, -JNK/SAPK. The representative blots from 3 individual analyses are shown.

FIGS. 7A-7B. AR inhibition prevents the expression of proliferation cell nuclear antigen (PCNA) in the lens epithelial cells of pig eye capsular bag. Pig eye capsular bags were prepared by capsulorhexis and incubated in DMEM with 10% FBS and 1% penicillin/streptomycin with or without (FIG. 7A) sorbinil (50 μM) and (FIG. 7B) fidarestat (30 μM) for 7 days in humidified incubator at 37° C. with 95% O₂ and 5% CO₂. At the end of incubation period, capsular bags were cut into four pieces and flat mounted on glass slides with cellular side facing up. The pieces of capsular bags were fixed with 4% paraformaldehyde. Subsequently specimens were immunostained with antibodies against PCNA and photographed using light microscopes. (A representative photomicrograph in both anterior and posterior capsule is shown (n=6); Magnification 200×). Sorb, sorbinil; fida, fidarestat.

FIGS. 8A-8F. AR inhibition prevents the expression of differentiation markers in the lens epithelial cells of pig eye capsular bag. Pig eye capsular bags were prepared by capsulorhexis and incubated in DMEM with 10% FBS and 1% penicillin/streptomycin with or without (FIGS. 8A,C,E) sorbinil (50 μM) and (FIGS. B,D,F) fidarestat (30 μM) for 7 days in humidified incubator at 37° C. with 95% O₂ and 5% CO₂. At the end of incubation period, capsular bags were cut into four pieces and flat mounted on glass slides with cellular side facing up. The pieces of capsular bags were fixed with 4% paraformaldehyde. Subsequently specimens were immunostained with antibodies against (FIGS. 8A-B) α-SMA, (FIGS. 8C-D) β-crystallin and (FIGS. 8E-F) ICAM-1 and photographed using fluorescence and light microscopes, respectively. (A representative photomicrograph is shown (n=3); Magnification 200×). Sorb, sorbinil; fida, fidarestat.

FIGS. 9A-9D. AR inhibition prevents the expression of differentiation markers in the lens epithelium of pig eye capsular bags. Approximately 1×10⁵ cells were seeded on chambered slides. The cells were starved in 0.5% FBS medium with or without AR inhibitors for overnight. The cells were treated with b-FGF (50 ng/ml) for 24 h. Subsequently, the cells were washed with cold PBS (pH 7.2) and fixed in methanol pre-chilled at −20° C. The cells were washed with PBS again and immunostained using antibodies against (FIG. 9A) α-SMA, (FIG. 9B) β-crystallin, (FIG. 9C) filensin, and (FIG. 9D) ICAM-1. The slides were mounted with floursave (with DAPI) mounting medium. A fluorescence microscope (Nikon) was used for the acquisition of images. (A representative photomicrograph is shown (n=3); Magnification 200×). C, control; S, sorbinil, Z, zopolrestat; F, b-FGF.

### DETAILED DESCRIPTION OF THE INVENTION

The inventors have demonstrated that Aldose Reductase (AR) is important for the detoxification of lipid aldehydes. In addition to the detoxification role, AR activity is necessary for cell signaling of cytokines, chemokines, endotoxins, high glucose, and growth factors that cause cell apoptosis and proliferation which cause tissue dysfunction leading to inflammation and various diseases, i.e., AR is an obligatory mediator of cytokine, chemokine, growth factors, and bacterial endotoxin-induced by activation of transcription factors NF-κB and AP1 through a cascade of kinases. The activation of transcription factors is responsible for the synthesis and release of a number of cytokines, chemokines, and growth factors which cause cytotoxicity. They are responsible for causing inflammation in general (see U.S. patent application Ser. No. 11/210,283, filed Aug. 23, 2005; U.S. patent application Ser. No. 10/462,223, filed on Jun. 13, 2004; U.S. Provisional Application 60/603,725 filed on Aug. 23, 2004; U.S. Provisional Application 60/388,213, filed on Jun. 13, 2003, all of which are hereby incorporated by reference).

### I. ALDOSE REDUCTASE AND INFLAMMATORY BOWEL DISEASE

In certain aspects of the invention, aldose reductase inhibitor may be used to treat inflammatory bowel disease. In medicine, inflammatory bowel disease (IBD) is a group of inflammatory conditions of the large intestine and, in some cases, the small intestine. The main forms of IBD are Crohn's disease and ulcerative colitis (UC). Non-limiting examples of other forms of IBD are: Collagenous colitis, Lymphocytic colitis, Ischaemic colitis, Diversion colitis, Behçet's syndrome, Infective colitis and Indeterminate colitis.

A. Diagnosis

Although very different diseases, both Crohn's disease and ulcerative colitis may present with any of the following symptoms: abdominal pain, vomiting, diarrhea, hematochezia, weight loss and various associated complaints or diseases (arthritis, pyoderma gangrenosum, primary sclerosing cholangitis).

The diagnosis of IBD is based on a combination of exams: endoscopic, X-rays, and blood and tissue tests. Upon diagnosis, IBD patients may need additional tests to monitor the disease and diagnose possible complications or side effects of medications. Mayo specialists are very experienced at conducting and interpreting these tests.

In laboratory tests, a CBC (complete blood count) test can detect infection and anemia, as well as monitor for side effects of certain IBD medications. Liver function tests help screen for liver and bile duct abnormalities seen in some IBD patients. Stool studies determine whether patients have treatable bacterial infections that can trigger a flare-up of IBD. Antibody tests can help clarify the situation for “indeterminate colitis” patients without a definite diagnosis.

Several types of endoscopy are used to determine if the patient has ulcerative colitis or Crohn's disease and how much bowel is affected. All use a thin, flexible tube with a lighted camera inside the tip, which allows doctors to look at the lining of the gastrointestinal (GI) tract. The image is magnified and appears on a television screen. Each procedure is named for the part of the GI tract examined: Sigmoidoscopy—Examines the lining of the lower third of the large intestine (the sigmoid colon); Colonoscopy—Examines the lining of the large intestine (colon), and sometimes can peek into the very end of the small intestine (or ileum); EGD (Esophagogastroduodenoscopy)—Examines the lining of the esophagus, stomach (gastro), and duodenum (first part of the small intestine); ERCP (Endoscopic retrograde cholangiopancreatography)—Examines the bile ducts in the liver and the pancreatic duct; Endoscopic ultrasound—Uses an ultrasound probe attached to an endoscope to obtain deep images of the gut. In IBD, this is most often used to look at fistulas in the rectal area; Capsule endoscopy—Patients swallow computerized cameras in vitamin-sized capsules to produce images of sections of the small intestine that are beyond the reach of an EGD. Read more on capsule endoscopy.

Radiologic tests provide information that endoscopy cannot. EGD and colonoscopy can visualize only the stomach, the very upper small intestine (EGD) and the colon and very lower small intestine (colonoscopy). Most of the small intestine cannot be imaged by endoscopy, although Mayo is currently evaluating capsule endoscopy for this purpose. Radiographic tests can image the small intestine.

Plain X-rays without contrast detect blockage in the small or large intestine. Contrast X-rays are used with endoscopy in monitoring and treating IBD. These X-rays track special liquid contrast, usually barium, as it passes through the intestine, highlighting specific conditions. A CT scanner takes simultaneous X-rays from different angles to reconstruct images of the internal organs. Magnetic Resonance Imaging (MRI) is used to evaluate perianal fistulas and abscesses in patients with IBD. Other potential uses are being investigated. Inflammation of the GI tract is characteristic of ulcerative colitis and Crohn's disease. Leukocyte scintigraphy (tagged white blood cell scan) detects white blood cell accumulation in inflamed tissue. In general, ultrasound technology is not useful for examining the bowel, although sometimes it is used in combination with other radiological tests.

B. Treatment

Depending on the level of severity, IBD may require immunosuppression to control the symptoms. such as azathioprine, methotrexate, or 6-mercaptopurine. More commonly, treatment of IBD requires a form of mesalamine. Often, steroids are used to control disease flares and were once acceptable as a maintenance drug. In use for several years in Crohn's disease patients and recently in patients with Ulcerative Colitis, biologicals has been used such as the intravenously administered Remicade. Severe cases may require surgery, such as bowel resection, strictureplasty or a temporary or permanent colostomy or ileostomy. Alternative medicine treatments for bowel disease exist in various forms, however such methods concentrate on controlling underlying pathology in order to avoid prolonged steroidal exposure or surgical excisement.

Usually the treatment is started by administering drugs with high anti-inflammatory affects, such as Prednisone. Once the inflammation is successfully controlled, the patient is usually switched to a lighter drug to keep the disease in remission, such as Asacol, a mesalamine. If unsuccessful, a combination of the aforementioned immunosurpression drugs with a mesalamine (which may also have an anti-inflammatory effect) may or may not be administered, depending on the patient. While IBD can limit quality of life due to pain, vomiting, diarrhea, and other socially unacceptable symptoms, it is rarely fatal on its own. Fatalities due to complications such as toxic megacolon, bowel perforation and surgical complications are also rare.

C. Prognosis

While patients of IBD do have an increased risk of colorectal cancer this is usually caught much earlier than the general population in routine surveillance of the colon by colonoscopy, and therefore patients are much more likely to survive.

The goal of treatment is toward achieving remission, after which the patient is usually switched to a lighter drug with fewer potential side effects. Every so often an acute resurgence of the original symptoms may appear: this is known as a “flare-up”. Depending on the circumstances, it may go away on its own or require medication. The time between flare-ups may be anywhere from weeks to years, and varies wildly between patients—a few have never experienced a flare-up.

D. IBD and Inflammation

The inflammatory bowel diseases (IBD) are characterized by chronic inflammation of the gastrointestinal tract. The current hypotheses suggest that persistent intestinal inflammation may be the result of either enhanced or aberrant immunologic responsiveness to normal constituents of the gut lumen or an overall autoimmune dysregulation and imbalance. The number of people being diagnosed with IBD has increased as the number of infections by parasites, such as roundworm, hookworm and human whipworms, has fallen, and the condition is still rare in countries where parasitic infections are common. This is similar to the hygiene hypothesis applied to allergies

The recent establishment of various animal models relevant for studying the pathogenesis of intestinal inflammation has provided some insight into potential disease mechanisms. There is increasing evidence that the intestinal microflora is an important cofactor in the pathogenesis of intestinal inflammation. Epithelial cells from intestine, gut and colon play a major role in providing a barrier that regulates contact between bacteria and immune cells. Increased inflammatory response in the epithelial cells due to increased levels of cytokines, growth factors and chemokines contribute to the cytotoxicity leading to tissue damage and dysfunction as observed in IBD.

### II. ALDOSE REDUCTASE AND MACULAR DEGENERATION

The inventors have the data to show that in macular degeneration aldehydes content increases and aldehyde-protein adducts are formed. Since cytokines have been shown to be involved in AMD (age-related macular degeneration), and the inventors found that AR inhibitors prevent the increase as well as effect of cytokines, AR inhibitors are used for the therapy of AMD in certain aspects of the present invention.

Macular degeneration is a medical condition predominantly found in young children in which the center of the inner lining of the eye, known as the macula area of the retina, suffers thickening, atrophy, and in some cases, watering. This can result in loss of side vision, which entails inability to see coarse details, to read, or to recognize faces. According to the American Academy of Opthalmology, it is the leading cause of central vision loss (blindness) in the United States today for those under the age of twenty years. Although some macular dystrophies that affect younger individuals are sometimes referred to as macular degeneration, the term generally refers to age-related macular degeneration (AMD or ARMD).

Age-related macular degeneration begins with characteristic yellow deposits in the macula (central area of the retina which provides detailed central vision, called fovea) called drusen between the retinal pigment epithelium and the underlying choroid. Most people with these early changes (referred to as age-related maculopathy) have good vision. People with drusen can go on to develop advanced AMD. The risk is considerably higher when the drusen are large and numerous and associated with disturbance in the pigmented cell layer under the macula. Recent research suggests that large and soft drusen are related to elevated cholesterol deposits and may respond to cholesterol lowering agents or the Rheo Procedure.

Advanced AMD, which is responsible for profound vision loss, has two forms: dry and wet. Central geographic atrophy, the dry form of advanced AMD, results from atrophy to the retinal pigment epithelial layer below the retina, which causes vision loss through loss of photoreceptors (rods and cones) in the central part of the eye. While no treatment is available for this condition, vitamin supplements with high doses of antioxidants, lutein and zeaxanthin, have been demonstrated by the National Eye Institute and others to slow the progression of dry macular degeneration and in some patients, improve visual acuity.

Neovascular or exudative AMD, the wet form of advanced AMD, causes vision loss due to abnormal blood vessel growth in the choriocapillaries, through Bruch's membrane, ultimately leading to blood and protein leakage below the macula. Bleeding, leaking, and scarring from these blood vessels eventually cause irreversible damage to the photoreceptors and rapid vision loss if left untreated.

Until recently, no effective treatments were known for wet macular degeneration. However, new drugs, called anti-angiogenics or anti-VEGF (anti-Vascular Endothelial Growth Factor) agents, when injected directly into the vitreous humor of the eye using a small, painless needle, can cause regression of the abnormal blood vessels and improvement of vision. The injections frequently have to be repeated on a monthly or bi-monthly basis. Examples of these agents include Lucentis, Avastin and Macugen. Only Lucentis and Macugen are FDA approved as of April 2007. Macugen has been found to have only minimal benefits in neovascular AMD and is no longer used. Worldwide, Avastin has been used extensively despite its “off label” status. The cost of Lucentis is approximately $2000 US per treatment while the cost of Avastin is approximately $150 per treatment. Lucentis is a close chemical relative of Avastin. Both drugs are made by Genentech.

### III. ALDOSE REDUCTASE AND MACULAR EDEMA

In certain aspects of the invention, aldose reductase inhibitor may be used to treat macular edema. Macular edema is due to oxidative stress and NFκb activation is involved Macular edema occurs when fluid and protein deposits collect on or under the macula of the eye, a yellow central area of the retina, causing it to thicken and swell. The swelling may distort a person's central vision, as the macula is near the center of the retina at the back of the eyeball. This area holds tightly packed cones that provide sharp, clear central vision to enable a person to see form, color, and detail that is directly in the line of sight.

Cystoid macular edema (CME) is a type of macular edema that includes cyst formation. Cystoid macular edema is a swelling of the macula which most commonly presents after routine, uncomplicated cataract surgery. The condition presents with decreased visual acuity, most commonly 4 to 12 weeks following surgery. Sometimes the condition presents as early as a few days, or as late as many months, after surgery.

CME typically presents with a complaint of painless visual loss in one eye. It can be bilateral, depending on the etiology. The onset of symptoms is usually gradual; however, patients often only notice it suddenly, when they check one eye separately. Different causes of CME have different clinical presentations. The most common entities are discussed below.

Diabetic maculopathy affects the capillaries in the macular region, leading to macular edema. Occasionally, a CME component of the macular edema develops, with cystoid changes in the foveal region. This is more common in cases of diffuse and chronic diabetic macular edema, and the vision may be reduced to the 20/200 level. When eyes with clinically significant macular edema (ie, edema overwhelming the homeostasis of the retina causing noticeable thickening) are treated early, before the onset of diffuse edema, CME possibly can be avoided if the patient maintains excellent control of the underlying medical problems.

CME, in association with diabetic macular edema, has also been correlated to the presence of an attached posterior hyaloid, whereas patients with a posterior vitreous separation are much less likely to develop a component of CME. This may support a mechanical mechanism of the development of CME, where tractional forces induce the formation of cystoid spaces in the macula. Alternatively, the traction on the macula may lift the retina away from the RPE pump, causing CME. Occasionally, even in the absence of an attached posterior hyaloid, a preretinal membrane can exert tractional forces and lead to CME.

Age-related macular degeneration (ARMD) can present in 1 of 2 forms: atrophic or exudative (dry or wet). Atrophic macular degeneration without exudative changes does not generally lead to CME. The exudative form of ARMD, with choroidal neovascularization, can cause a serous detachment of the overlying retina and resultant CME. CME is more common if the serous detachment of the macula has been present for 3-6 months or if the choroidal neovascular membrane has involved most of the subfoveal region. In such cases, the likelihood of restoring good vision is low.

Retinal vein occlusion, a branch retinal vein occlusion (BRVO), or a central retinal vein occlusion (CRVO) can cause macular edema resulting from breakdown of the capillary endothelium associated with increased intravascular hydrostatic pressure. The damaged vessels leak fluid into the intercellular spaces, and, eventually, intraretinal cystoid spaces can be seen. This form of CME can be associated with further visual loss and usually results in some permanent visual loss if the situation persists for more than 6 months. However, it can improve with earlier resolution of the macular edema.

Recovery of the macular edema can occur after laser therapy (even if the edema persists >6 mo) or with development of collateral vessels. Although laser grid photocoagulation has been shown to improve visual outcome in patients with BRVO, patients with CRVO do not appear to benefit from laser photocoagulation to treat macular edema. Although the edema may resolve, the visual outcome is unchanged.

Diabetic macular edema is swelling of the retina in diabetes mellitus due to leaking of fluid from blood vessels within the macula. As macular edema develops, blurring occurs in the middle or just to the side of the central visual field. Visual loss from diabetic macular edema can progress over a period of months and make it impossible to focus clearly.

Macular edema in common in diabetes. The lifetime risk for diabetics to develop macular edema is about 10%. The condition is closely associated with the degree of diabetic retinopathy (retinal disease). Hypertension (high blood pressure) and fluid retention also increase the hydrostatic pressure within capillaries which drives fluid from within the vessels into the retina. A common cause of fluid retention in diabetes is kidney disease with loss of protein in the urine (proteinuria).

Diabetic macular edema is classified into focal and diffuse types. This is an important difference because the two types differ in treatment. Focal macular edema is caused by foci of vascular abnormalities, primarily microaneurysms, which tend to leakage fluid whereas diffuse macular edema is caused by dilated retinal capillaries in the retina.

Two types of laser treatment for diabetic macular edema are focal and grid. Focal laser treatment is used to treat focal diabetic macular edema; the aim is to close leaking microaneurysms. Grid laser treatment is used to treat diffuse diabetic macular edema and is applied to areas of retinal thickening in which there is diffuse leakage; the aim is to produce a retinal burn of mild to moderate intensity. The patient is rechecked several months after treatment and, if the diabetic macular edema is not responding to treatment, the laser treatment is repeated. The goal of treatment is to maintain current visual acuity and reduce the chances of progressive visual loss. Even with successful treatment, visual acuity often does not improve.

Newer approaches to treating macular edema associated with retinal vein occlusions include intraocular steroids and vascular endothelial growth factor (VEGF) inhibitors. Macular edema may be due to oxidative stress and NFrb activation may be involved, suggesting a new approach to discover new candidates for therapeutic treatment.

### IV. ALDOSE REDUCTASE AND POSTERIOR CAPSULE OPACIFICATION

Posterior capsular opacification (PCO), also called secondary cataract, is the most common postoperative complication of cataract surgery that results in loss of vision. Decrease in visual acuity due to PCO occurs in approximately 30% of subjects who undergo phacoemulsification surgery (Apple et al., 1992; Ohadi et al., 1991) with younger patients being at higher risk. PCO develops due to proliferation and differentiation and/or trans-differentiation of residual lens epithelial cells (LEC) at the equator and beneath the anterior lens capsule after cataract surgery. The residual lens epithelial cells proliferate and migrate on top of the posterior capsule underneath the intraocular lens as well causing the obstruction in the vision. Many of these cells undergo epithelial mesenchymal transition (EMT), a change in phenotype from an epithelial to fibrocytic morphology accompanied by aberrant basement membrane synthesis, and differentiation unusually resulting in the formation of fibroblasts and spindle like myofibroblasts, causing opacification (De Iongh et al., 2005). Therefore, to prevent PCO, post surgical pharmacological inhibition of LEC proliferation, migration, and EMT is a potential option. In certain aspects, an ARI can be impregnated in, coated onto, or coupled to lens replacement materials. Inventors contemplate various methods to attach or couple ARI to a surgical implant or surgical material so that ARI is released over time in amount sufficient to suppress opacification. In this context, several drugs that can block LEC proliferation, migration, and EMT have been studied, but to date none of them have been clinically effective (Seki et al., 1992; Yabe, Kawashima Long-term efficacy of diclofenac sodium after PEA and IOL Cortina et al., 1997; Nishi et al., 1999; Biswas et al., 1999).

Many studies have suggested the role of several cytokines and growth factors such as IL-6 (Biswas et al., 1999, transforming growth factor (TGF)-p (Ohadi et al., 1991; Meacock et al., 2000), fibroblast growth factor (FGF)-2 (Liu et al., 1994; Mansfield et al., 2004), hepatocyte growth factor (HGF) (Symonds et al., 2006; Wormstone et al., 2000), and epidermal growth factor (EGF) (Choi et al., 2004) in the development of PCO. Subsequent to cataract surgery, the levels of these cytokines and growth factors are elevated in aqueous humor influencing proliferation, migration and trans-differentiation of LEC. Basic-FGF or FGF-2 (b-FGF), an important growth factor that establishes and maintains normal lens physiology, is constantly present in the normal lens milieu. It has been shown that b-FGF regulates proliferation, and promotes the differentiation of epithelial cells to fiber cells (Jiang et al., 2006). De Iongh et al. (2005) have suggested that like b-FGF, TGF-β could also induce an aberrant fiber cell differentiation causing the LEC to undergo EMT and differentiation into fibroblastic or myofibroblastic cells that could be contributing to lens pathology. Studies have shown that b-FGF induces LEC proliferation at low doses and differentiation at higher doses, which may contribute to the development of PCO (Ohadi et al., 1991). Though the exact mechanism involved in b-FGF-induced PCO development is not known, several studies have presented evidence showing the role of b-FGF-stimulated activation of ERK1/2, and expression of differentiation markers such as α-smooth muscle actin (α-SMA), β-crystallin, and filensin which may cause EMT and trans-differentiation in PCO (Lang and McAvoy, 2004; Lovicu and McAvoy, 2001).

On the other hand TGF-β induces an epithelial mesenchymal transition (EMT) causing the residual LEC to trans-differentiate into myofibroblastic cells (De Iongh et al., 2005; Lee et al., 1999). Hales et al (1995) showed that TGF-β-induced opacification in the cultured lens was morphologically and biochemically similar to cataract. Immediately after cataract surgery, elevated levels of b-FGF and low levels of active TGF-β may induce LEC to proliferate. However, when the level of active TGF-β increases it inhibits b-FGF-induced proliferation and stimulates PCO-specific changes such as EMT and transdifferentiation (Meacock et al., 2000).

Several reports also suggest that growth factors-induced ROS formation mediates molecular signaling leading to lens epithelial cell growth and differentiation that may cause PCO (Choi et al., 2004; Iyengar et al., 2007). Further, antioxidants such as caffeic acid, and retinoic acids have been shown to prevent PCO (Chen et al., 2007; Hepsen et al., 1997; Doganay et al., 2002).

The inventors have shown recently that aldose reductase (AR), a polyol pathway enzyme that reduces glucose and various lipid peroxidation derived aldehydes and their glutathione conjugates, mediates ROS signals initiated by growth factors, cytokines, high glucose and bacterial endotoxin (Lypopolysaccheride; LPS) (Inan et al., 2001; Ramana et al., 2003; Pladzyk et al., 2006; Ramana et al., 2006). Furthermore, they have shown that pharmacological inhibition or genetic (siRNA and antisense) ablation of AR prevents activation of ROS-sensitive transcription factors such as NF-κB and AP-1 in human lens epithelial cells, and macrophages (Inan et al., 2001; Ramana et al., 2003; Pladzyk et al., 2006). They have also shown that inhibition of AR prevents the expression of TNF-α, iNOS, Cox-2 in LPS-induced HLEC and rat eyes (Ramana et al., 2003; Tammali et al., 2006). Indeed, the association of AR with ROS-mediated signaling is evident by the observations that AR inhibition attenuates high glucose-induced oxidative stress and superoxide production in LEC and retinal pericytes (Inan et al., 2001; Yadav et al., 2007).

Further, AR has also been implicated in the development of diabetic cataractogenesis (DC) due to 1) increased flux of glucose through the polyol pathway that increases osmotic pressure, 2) decreased NADPH/NADH ratio that could increase oxidative stress, 3) activation of PKC which activates ROS signals and 4) increased advanced glycation end products formation leading to protein alteration and tissue damage (Cheung et al., 2005). The inventors and others have shown that inhibition of AR prevents DC in rodents (Sheetz and King, 2002; Zenon et al., 1990; Lee and Chung, 1999). Further, AR has been found to be growth response protein and over-expressed during oxidative stress, diabetes, cancer and in the cells undergoing proliferation in vitro as well as in vivo (Srivastava et al., 2005; ramana et al., 2002).

The inventors contemplate that AR could play a major role in the development of PCO and inhibition of AR could be a therapeutic target to prevent PCO. Here the inventors have investigated the role of AR in mediation of LEC growth and trans-differentiation in cultured HLEC and in cultured pig eye capsular bags. Their results show that inhibition of AR prevents b-FGF-induced LEC growth and transdifferentiation in vitro in cultured HLEC and in pig eye capsular bags explants suggesting a potential therapeutic use of AR inhibitors in the prevention of PCO.

An effective and common treatment for cataracts is to surgically remove the cloudy lens. There are two types of surgery that can be used to remove cataracts: extra-capsular (extracapsular cataract extraction, or ECCE) and intra-capsular (intracapsular cataract extraction, or ICCE). Extra-capsular (ECCE) surgery consists of removing the lens but leaving the majority of the lens capsule intact. High frequency sound waves (phacoemulsification) are sometimes used to break up the lens before extraction. Intra-capsular (ICCE) surgery involves removing the entire lens of the eye, including the lens capsule, but it is rarely performed in modern practice. In either extra-capsular surgery or intra-capsular surgery, the cataractous lens is removed and replaced with a plastic lens (an intraocular lens implant) which stays in the eye permanently. Each of the surgeries can be used in combination with administration of ARIs to the patient, before, during and/after surgery.

Cataract operations are usually performed using a local anaesthetic and the patient is allowed to go home the same day. Recent improvements in intraocular technology now allow cataract patients to choose a multifocal lens to create a visual environment in which they are less dependent on glasses. Traditional intraocular lenses are monofocal.

Complications after cataract surgery, including endophthalmitis, posterior capsular opacification and retinal detachment, are possible. In certain aspects these complications can be treated, reduced, or prevented by administering aldose reductase inhibitors (ARI)

### V. ALDOSE REDUCTASE INHIBITORS

The inhibitors of aldose reductase can be any compound that inhibits the enzyme aldose reductase. The aldose reductase inhibitor compounds of this invention are readily available or can be easily synthesized by those skilled in the art using conventional methods of organic synthesis, particularly in view of the pertinent patent specifications.

Many of these are well known to those of skill in the art, and a number of pharmaceutical grade AR inhibitors are commercially available, such as fidarestat (SNK-860), (2S,4S)-2-aminoformyl-6-fluoro-spiro[chroman-4,4′-imidazolidine]-2′,5′-dione (CAS number 136087-85-9); Tolrestat, N-[[6-methoxy-5-(trifluoromethyl)-1-naphthalenyl]thioxomethyl]-N-methylglycine, [Wyeth-Ayerst, Princeton, N.J.; other designations are Tolrestatin, CAS Registry Number 82964-04-3, Drug Code AY-27,773, and brand names ALREDASE (Am. Home) and LORESTAT (Recordati)]; Ponalrestat, 3-(4-bromo-2-fluorobenzyl)-4-oxo-3H-phthalazin-1-ylacetic acid [ICI, Macclesfield, U.K.; other designations are CAS Registry Number 72702-95-5, ICI-128,436, and STATIL (ICI)]; Sorbinil, (S)-6-fluoro-2,3-dihydrospiro[4H-1-benzopyran-4,4′-imidazolidine]-2′,5′-di one (Pfizer, Groton, Conn.; CAS Registry Number 68367-52-2, Drug Code CP-45,634); EPALRESTAT (ONO, Japan); METHOSORBINIL (Eisal); ALCONIL (Alcon); AL-1576 (Alcon); CT-112 (Takeda); and AND-138 (Kyorin).

Other ARIs have been described. For a review of ARIs used in the diabetes context, see Humber, “Aldose Reductase Inhibition: An Approach to the Prevention of Diabetes Complications”, Porte, ed., Ch. 5, pp. 325-353; Tomlinson et al., 1992), such as spirohydantoins and related structures, spiro-imidazolidine-2′,5′-diones; and heterocycloic alkanoic acids. Other aldose reductase inhibitors are ONO-2235; zopolrestat; SNK-860; 5-3-thienyltetrazol-1-yl (TAT); WAY-121,509; ZENECA ZD5522; M16209; (5-(3′-indolal)-2-thiohydantoin; zenarestat; zenarestat 1-O-acylglucuronide; SPR-210; (2S,4S)-6-fluoro-2′,5′-dioxospiro-[chroman-4,4′-imidazolidine]-2-carboxamide (SNK-880); arylsulfonylamino acids; 2,7-difluorospirofluorene-9,5′-imidazolidine-2′,4′-dione (imiriestat, Al11576, HOE 843); and isoliquiritigenin.

In some embodiments, the aldose reductase inhibitor is a compound that directly inhibits the bioconversion of glucose to sorbitol catalyzed by the enzyme aldose reductase. Such aldose reductase inhibitors are direct inhibitors, which are contemplated as part of the invention. Direct inhibition is readily determined by those skilled in the art according to standard assays (Malone, 1980). The following patents and patent applications, each of which is hereby wholly incorporated herein by reference, exemplify aldose reductase inhibitors which can be used in the compositions, methods and kits of this invention, and refer to methods of preparing those aldose reductase inhibitors: U.S. Pat. Nos. 4,251,528; 4,600,724; 4,464,382, 4,791,126, 4,831,045; 4,734,419; 4,883,800; 4,883,410; 4,883,410; 4,771,050; 5,252,572; 5,270,342; 5,430,060; 4,130,714; 4,540,704; 4,438,272; 4,436,745, 4,438,272; 4,436,745, 4,438,272; 4,436,745, 4,438,272; 4,980,357; 5,066,659; 5,447,946; and 5,037,831.

A variety of aldose reductase inhibitors are specifically described and referenced below, however, other aldose reductase inhibitors will be known to those skilled in the art. Also, common chemical names or other designations are in parentheses where applicable, together with reference to appropriate patent literature disclosing the compound. Accordingly, examples of aldose reductase inhibitors useful in the compositions, methods and kits of this invention include, but are not limited to: 3-(4-bromo-2-fluorobenzyl)-3,4-dihydro-4-oxo-1-phthalazineacetic acid (ponalrestat, U.S. Pat. No. 4,251,528); N[[(5-trifluoromethyl)-6-methoxy-1-naphthalenyl]thioxomethyl}-N-methylglycine (tolrestat, U.S. Pat. No. 4,600,724); 5-[(Z,E)-β-methylcinnamylidene]-4-oxo-2-thioxo-3-thiazolideneacetic acid (epalrestat, U.S. Pat. No. 4,464,382, U.S. Pat. No. 4,791,126, U.S. Pat. No. 4,831,045); 3-(4-bromo-2-fluorobenzyl)-7-chloro-3,4-dihydro-2,4-dioxo-[(2H)-quinazolineacetic acid (zenarestat, U.S. Pat. No. 4,734,419, and U.S. Pat. No. 4,883,800); 2R,4R-6,7-dichloro-4-hydroxy-2-methylchroman-4-acetic acid (U.S. Pat. No. 4,883,410); 2R,4R-6,7-dichloro-6-fluoro-4-hydroxy-2-methylchroman-4-acetic acid (U.S. Pat. No. 4,883,410); 3,4-dihydro-2,8-diisopropyl-3-oxo-2H-1,4-benzoxazine-4-acetic acid (U.S. Pat. No. 4,771,050); 3,4-dihydro-3-oxo-4-[(4,5,7-trifluoro-2-benzothiazolyl)methyl]-2H-1,4-benzothiazine-2-acetic acid (SPR-210, U.S. Pat. No. 5,252,572); N-[3,5-dimethyl-4-[(nitromethyl)sulfonyl]phenyl]-2-methyl-benzeneacetamide (ZD5522, U.S. Pat. No. 5,270,342 and U.S. Pat. No. 5,430,060); (S)-6-fluorospiro[chroman-4,4′-imidazolidine]-2,5′-dione (sorbinil, U.S. Pat. No. 4,130,714); d-2-methyl-6-fluoro-spiro(chroman-4′,4′-imidazolidine)-2′,5′-dione (U.S. Pat. No. 4,540,704); 2-fluoro-spiro(9H-fluorene-9,4′-imidazolidine)-2′,5′-dione (U.S. Pat. No. 4,438,272); 2,7-di-fluoro-spiro(9H-fluorene-9,4′-imidazolidine)-2′,5′-dione (U.S. Pat. No. 4,436,745, U.S. Pat. No. 4,438,272); 2,7-di-fluoro-5-methoxy-spiro(9H-fluorene-9,4′-imidazolidine)-2′,5′-dione (U.S. Pat. No. 4,436,745, U.S. Pat. No. 4,438,272); 7-fluoro-spiro(5H-indenol[1,2-b]pyridine-5,3′-pyrrolidine)-2,5′-dione (U.S. Pat. No. 4,436,745, U.S. Pat. No. 4,438,272); d-cis-6′-chloro-2′,3′-dihydro-2′-methyl-spiro-(imidazolidine-4,4′-4′H-pyrano(2,3-b)pyridine)-2,5-dione (U.S. Pat. No. 4,980,357); spiro[imidazolidine-4,5′(6H)-quinoline]-2,5-dione-3′-chloro-7,′8′-dihydro-7′-methyl-(5′-cis) (U.S. Pat. No. 5,066,659); (2S,4S)-6-fluoro-2′,5′-dioxospiro(chroman-4,4′-imidazolidine)-2-carboxamide (fidarestat, U.S. Pat. No. 5,447,946); and 2-[(4-bromo-2-fluorophenyl)methyl]-6-fluorospiro[isoquinoline-4(1H), 3′-pyrrolidine]-1,2′, 3,5′(2H)-tetrone (minalrestat, U.S. Pat. No. 5,037,831). Other compounds include those described in U.S. Pat. Nos. 6,720,348, 6,380,200, and 5,990,111, which are hereby incorporated by reference. Moreover, in other embodiments it is specifically contemplated that any of these may be excluded as part of the invention.

Embodiments of the invention contemplate inhibitors of aldose reductase that are peptides or proteins that form a proteinaceous composition. It is contemplated that any teaching with respect to one particular proteinaceous composition may apply generally to other proteinaceous compositions described herein.

As used herein, a “proteinaceous molecule,” “proteinaceous composition,” “proteinaceous compound,” or “proteinaceous material” generally refers, but is not limited to, a protein of greater than about 200 amino acids or the full length endogenous sequence translated from a gene; a polypeptide of greater than about 100 amino acids; and/or a peptide of from about 3 to about 100 amino acids. All the “proteinaceous” terms described above may be used interchangeably herein.

In certain embodiments of the invention, the proteinaceous composition may include such molecules that may comprise, but is not limited to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 383, 385 or greater amino molecule residues, and any range derivable therein. Such lengths are applicable to all polypeptides and peptides mentioned herein. It is contemplated that an aldose reductase inhibitor may specifically bind or recognize a particular region of AR, including 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 383, 385 or greater contiguous amino acids of aldose reductase or any range of numbers of contiguous amino acids derivable therein. Aldose reductase may be from any organism, including mammals, such as a human, monkey, mouse, rat, hamster, cow, pig, rabbit, and may be from other cultured cells readily available. AR inhibitors may also affect polypeptides in pathways involving AR but found further upstream or downstream from AR in the pathway.

It will also be understood that amino acid sequences or nucleic acid sequences of AR, AR polypeptide inhibitors, or screening proteins may include additional residues, such as additional N- or C-terminal amino acids or 5′ or 3′ sequences, or various combinations thereof, and yet still be essentially as set forth in one of the sequences disclosed herein, so long as the sequence meets the criteria set forth above, including the maintenance of biological protein, polypeptide or peptide activity where expression of a proteinaceous composition is concerned. The addition of terminal sequences particularly applies to nucleic acid sequences that may, for example, include various non-coding sequences flanking either of the 5′ and/or 3′ portions of the coding region or may include various internal sequences, i.e., introns, which are known to occur within genes.

Another embodiment for the preparation of polypeptides according to the invention is the use of peptide mimetics. Peptide mimetics may be screened as a candidate substance. Mimetics are peptide-containing compounds, that mimic elements of protein secondary structure. The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used, in conjunction with the principles outlined above, to engineer second generation molecules having many of the natural properties of AR inhibitors, but with altered and even improved characteristics.

The present invention also contemplates the synthesis of peptides that can directly or indirectly inhibit AR. Because of their relatively small size, the peptides of the invention can also be synthesized in solution or on a solid support in accordance with conventional techniques. Various automatic synthesizers are commercially available and can be used in accordance with known protocols. See, for example, Tam et al., (1983); Merrifield, (1986); and Barany and Merrifield (1979). Short peptide sequences, or libraries of overlapping peptides, usually from about 6 up to about 35 to 50 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

In one embodiment, nucleic acid sequences complementary to at least a portion of the nucleic acid encoding AR will find utility as AR inhibitors. The use of a probe or primer of between 13 and 100 nucleotides, preferably between 17 and 100 nucleotides in length, or in some aspects of the invention up to 1-2 kilobases or more in length, allows the formation of a duplex molecule that is both stable and selective. In certain embodiments, these probes consist of oligonucleotide fragments. Such fragments should be of sufficient length to provide specific hybridization to a RNA or DNA tissue sample. The sequences typically will be 10-20 nucleotides, but may be longer. Longer sequences, e.g., 40, 50, 100, 500 and even up to full length, are preferred for certain embodiments.

Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNAs, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs may include regions complementary to intron/exon splice junctions. Thus, antisense constructs with complementarity to regions within 50-200 bases of an intron-exon splice junction may be used. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme) could be designed. These molecules, though having less than 50% homology, would bind to target sequences under appropriate conditions.

The use of AR-specific ribozymes is claimed in the present application. The following information is provided in order to compliment the earlier section and to assist those of skill in the art in this endeavor. Ribozymes are RNA-protein complexes that cleave nucleic acids in a site-specific fashion. Ribozymes have specific catalytic domains that possess endonuclease activity (Kim and Cech, 1987; Forster and Symons, 1987). For example, a large number of ribozymes accelerate phosphoester transfer reactions with a high degree of specificity, often cleaving only one of several phosphoesters in an oligonucleotide substrate (Cech et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence specific cleavage/ligation reactions involving nucleic acids (Joyce, 1989; Cech et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990; Sioud et al., 1992). In light of the information included herein and the knowledge of one of ordinary skill in the art, the preparation and use of additional ribozymes that are specifically targeted to a given gene, e.g., AR gene, will now be straightforward.

Several different ribozyme motifs have been described with RNA cleavage activity (reviewed in Symons, 1992). Examples that would be expected to function equivalently for the down regulation of AR include sequences from the Group I self splicing introns including tobacco ringspot virus (Prody et al., 1986), avocado sunblotch viroid (Symons, 1981), and Lucerne transient streak virus (Forster and Symons, 1987). Sequences from these and related viruses are referred to as hammerhead ribozymes based on a predicted folded secondary structure. Other suitable ribozymes include sequences from RNase P with RNA cleavage activity (Yuan et al., 1992, Yuan and Altman, 1994), hairpin ribozyme structures (Berzal-Herranz et al., 1992; Chowrira et al., 1993) and hepatitis δ virus based ribozymes (Perrotta and Been, 1992). The general design and optimization of ribozyme directed RNA cleavage activity has been discussed in detail (Haseloff and Gerlach, 1988, Symons, 1992, Chowrira, et al., 1994; Thompson, et al., 1995).

The other variable on ribozyme design is the selection of a cleavage site on a given target RNA. Ribozymes are targeted to a given sequence by virtue of annealing to a site by complimentary base pair interactions. Two stretches of homology are required for this targeting. These stretches of homologous sequences flank the catalytic ribozyme structure defined above. Each stretch of homologous sequence can vary in length from 7 to 15 nucleotides. The only requirement for defining the homologous sequences is that, on the target RNA, they are separated by a specific sequence which is the cleavage site. For hammerhead ribozymes, the cleavage site is a dinucleotide sequence on the target RNA, uracil (U) followed by either an adenine, cytosine or uracil (A,C or U; Perriman, et al., 1992; Thompson, et al., 1995). The frequency of this dinucleotide occurring in any given RNA is statistically 3 out of 16. Therefore, for a given target messenger RNA of 1000 bases, 187 dinucleotide cleavage sites are statistically possible.

Designing and testing ribozymes for efficient cleavage of a target RNA is a process well known to those skilled in the art. Examples of scientific methods for designing and testing ribozymes are described by Chowrira et al., (1994) and Lieber and Strauss (1995), each incorporated by reference. The identification of operative and preferred sequences for use in AR-targeted ribozymes is simply a matter of preparing and testing a given sequence, and is a routinely practiced “screening” method known to those of skill in the art.

An RNA molecule capable of mediating RNA interference in a cell is referred to as “siRNA.” Elbashir et al. (2001) discovered a clever method to bypass the anti viral response and induce gene specific silencing in mammalian cells. Several 21-nucleotide dsRNAs with 2 nucleotide 3′ overhangs were transfected into mammalian cells without inducing the antiviral response. The small dsRNA molecules (also referred to as “siRNA”) were capable of inducing the specific suppression of target genes.

In the context of the present invention, siRNA directed against AR, NF-κB, and TNF-α are specifically contemplated. The siRNA can target a particular sequence because of a region of complementarity between the siRNA and the RNA transcript encoding the polypeptide whose expression will be decreased, inhibited, or eliminated.

An siRNA may be a double-stranded compound comprising two separate, but complementary strands of RNA or it may be a single RNA strand that has a region that self-hybridizes such that there is a double-stranded intramolecular region of 7 basepairs or longer (see Sui et al., 2002 and Brummelkamp et al., 2002 in which a single strand with a hairpin loop is used as a dsRNA for RNAi). In some cases, a double-stranded RNA molecule may be processed in the cell into different and separate siRNA molecules.

In some embodiments, the strand or strands of dsRNA are 100 bases (or basepairs) or less, in which case they may also be referred to as “siRNA.” In specific embodiments the strand or strands of the dsRNA are less than 70 bases in length. With respect to those embodiments, the dsRNA strand or strands may be from 5-70, 10-65, 20-60, 30-55, 40-50 bases or basepairs in length. A dsRNA that has a complementarity region equal to or less than 30 basepairs (such as a single stranded hairpin RNA in which the stem or complementary portion is less than or equal to 30 basepairs) or one in which the strands are 30 bases or fewer in length is specifically contemplated, as such molecules evade a mammalian's cell antiviral response. Thus, a hairpin dsRNA (one strand) may be 70 or fewer bases in length with a complementary region of 30 basepairs or fewer.

Methods of using siRNA to achieve gene silencing are discussed in WO 03/012052, which is specifically incorporated by reference herein. Designing and testing siRNA for efficient inhibition of expression of a target polypeptide is a process well known to those skilled in the art. Their use has become well known to those of skill in the art. The techniques described in U.S. Patent Publication No. 20030059944 and 20030105051 are incorporated herein by reference. Furthermore, a number of kits are commercially available for generating siRNA molecules to a particular target, which in this case includes AR, NF-κB, and TNF-α. Kits such as Silencer™ Express, Silencer™ siRNA Cocktail, Silencer™ siRNA Construction, MEGAScript® RNAi are readily available from Ambion, Inc.

Other candidate AR inhibitors include aptamers and aptazymes, which are synthetic nucleic acid ligands. The methods of the present invention may involve nucleic acids that modulate AR, NF-κB, and TNF-α. Thus, in certain embodiments, a nucleic acid, may comprise or encode an aptamer. An “aptamer” as used herein refers to a nucleic acid that binds a target molecule through interactions or conformations other than those of nucleic acid annealing/hybridization described herein. Methods for making and modifying aptamers, and assaying the binding of an aptamer to a target molecule may be assayed or screened for by any mechanism known to those of skill in the art (see for example, U.S. Pat. Nos. 5,840,867, 5,792,613, 5,780,610, 5,756,291 and 5,582,981, Burgstaller et al., 2002, which are incorporated herein by reference.

### VI. PHARMACEUTICAL COMPOSITIONS AND ROUTES OF ADMINISTRATION

Pharmaceutical compositions of the present invention may comprise an effective amount of one or more AR inhibitors dissolved or dispersed in a pharmaceutically acceptable carrier to a subject. The phrases “pharmaceutical” or “pharmacologically acceptable” refers to molecular entities and compositions that do not produce an adverse, allergic or other untoward reaction when administered to an animal, such as, for example, a human, as appropriate. The preparation of a pharmaceutical composition that contains at least one AR inhibitor or additional active ingredient will be known to those of skill in the art in light of the present disclosure, and as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated. An AR inhibitor can be administered in the form of a pharmaceutically acceptable salt or with a pharmaceutically acceptable salt.

The expression “pharmaceutically acceptable salts” includes both pharmaceutically acceptable acid addition salts and pharmaceutically acceptable cationic salts, where appropriate. The expression “pharmaceutically-acceptable cationic salts” is intended to define, but is not limited to such salts as the alkali metal salts, (e.g., sodium and potassium), alkaline earth metal salts (e.g., calcium and magnesium), aluminum salts, ammonium salts, and salts with organic amines such as benzathine (N,N′-dibenzylethylenediamine), choline, diethanolamine, ethylenediamine, meglumine (N-methylglucamine), benethamine (N-benzylphenethylamine), diethylamine, piperazine, tromethamine (2-amino-2-hydroxymethyl-1,3-propanediol) and procaine. The expression “pharmaceutically-acceptable acid addition salts” is intended to define but is not limited to such salts as the hydrochloride, hydrobromide, sulfate, hydrogen sulfate, phosphate, hydrogen phosphate, dihydrogenphosphate, acetate, succinate, citrate, methanesulfonate (mesylate) and p-toluenesulfonate (tosylate) salts.

Pharmaceutically acceptable salts of the aldose reductase inhibitors of this invention may be readily prepared by reacting the free acid form of the aldose reductase inhibitor with an appropriate base, usually one equivalent, in a co-solvent. Typical bases are sodium hydroxide, sodium methoxide, sodium ethoxide, sodium hydride, potassium methoxide, magnesium hydroxide, calcium hydroxide, benzathine, choline, diethanolamine, piperazine and tromethamine. The salt is isolated by concentration to dryness or by addition of a non-solvent. In many cases, salts are preferably prepared by mixing a solution of the acid with a solution of a different salt of the cation (sodium or potassium ethylhexanoate, magnesium oleate), and employing a solvent (e.g., ethyl acetate) from which the desired cationic salt precipitates, or can be otherwise isolated by concentration and/or addition of a non-solvent.

The acid addition salts of the aldose reductase inhibitors of this invention may be readily prepared by reacting the free base form of said aldose reductase inhibitor with the appropriate acid. When the salt is of a monobasic acid (e.g., the hydrochloride, the hydrobromide, the p-toluenesulfonate, the acetate), the hydrogen form of a dibasic acid (e.g., the hydrogen sulfate, the succinate) or the dihydrogen form of a tribasic acid (e.g., the dihydrogen phosphate, the citrate), at least one molar equivalent and usually a molar excess of the acid is employed. However when such salts as the sulfate, the hemisuccinate, the hydrogen phosphate, or the phosphate are desired, the appropriate and exact chemical equivalents of acid will generally be used. The free base and the acid are usually combined in a co-solvent from which the desired salt precipitates, or can be otherwise isolated by concentration and/or addition of a non-solvent.

In addition, the aldose reductase inhibitors that may be used in accordance with this invention, prodrugs thereof, and pharmaceutically acceptable salts thereof or of said prodrugs, may occur as hydrates or solvates. These hydrates and solvates are also within the scope of the invention.

A pharmaceutical composition of the present invention may comprise different types of carriers depending on whether it is to be administered in solid, liquid or aerosol form, and whether it needs to be sterile for such routes of administration as injection. A pharmaceutical composition of the present invention can be administered intravenously, intradermally, intraarterially, intraperitoneally, intraarticularly, intrapleurally, intrabronchially, intrapleurally, intranasally, topically, intramuscularly, intraperitoneally, subcutaneously, subconjunctival, intravesicularlly, mucosally, intrapericardially, intraumbilically, orally, topically, locally, inhalation (e.g., aerosol inhalation), instillation, injection, infusion, continuous infusion, via a catheter, via a lavage, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The actual dosage amount of a composition of the present invention administered to a subject can be determined by physical and physiological factors such as body weight, severity of condition, the type of disease being treated, previous or concurrent therapeutic interventions, idiopathy of the patient and on the route of administration. The number of doses and the period of time over which the dose may be given may vary. The practitioner responsible for administration will, in any event, determine the concentration of active ingredient(s) in a composition and appropriate dose(s), as well as the length of time for administration for the individual subject. An amount of an aldose reductase inhibitor that is effective for inhibiting aldose reductase activity is used. Typically, an effective dosage for the inhibitors is in the range of about 0.01 mg/kg/day to 100 mg/kg/day in single or divided doses, preferably 0.1 mg/kg/day to 20 mg/kg/day in single or divided doses. Doses of about, at least about, or at most about 0.01, 0.05, 0.1, 0.15, 0.20, 0.25, 0.30, 0.35, 0.40, 0.45, 0.50, 0.55, 0.60, 0.65, 0.70, 0.75, 0.80, 0.85, 0.90. 0.95, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/kg/day, or any range derivable therein. Typically the dose will be 25 to 1200 mg per day and in certain aspects is between 100 and 800 mg per day.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

An AR inhibitor(s) of the present invention may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In certain aspects of the invention, the AR inhibitors are prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

In order to increase the effectiveness of treatments with the compositions of the present invention, such as an AR inhibitor, it may be desirable to combine it with other therapeutic agents. This process may involve contacting the cell(s) with an AR inhibitor and a therapeutic agent at the same time or within a period of time wherein separate administration of the modulator and an agent to a cell, tissue or organism produces a desired therapeutic benefit. The terms “contacted” and “exposed,” when applied to a cell, tissue or organism, are used herein to describe the process by which a AR inhibitor and/or therapeutic agent are delivered to a target cell, tissue or organism or are placed in direct juxtaposition with the target cell, tissue or organism. The cell, tissue or organism may be contacted (e.g., by administration) with a single composition or pharmacological formulation that includes both a AR inhibitor and one or more agents, or by contacting the cell with two or more distinct compositions or formulations, wherein one composition includes an AR inhibitor and the other includes one or more agents.

The AR inhibitor may precede, be concurrent with and/or follow the other agent(s) by intervals ranging from minutes to weeks. In embodiments where the AR inhibitor and other agent(s) are applied separately to a cell, tissue or organism, one would generally ensure that a significant period of time did not expire between the time of each delivery, such that the inhibitor and agent(s) would still be able to exert an advantageously combined effect on the cell, tissue or organism. For example, in such instances, it is contemplated that one may contact the cell, tissue or organism with two, three, four or more modalities substantially simultaneously (i.e., within less than about a minute) as the modulator. In other aspects, one or more agents may be administered within of from substantially simultaneously, about 1 minute, about 5 minutes, about 10 minutes, about 20 minutes about 30 minutes, about 45 minutes, about 60 minutes, about 2 hours, or more hours, or about 1 day or more days, or about 4 weeks or more weeks, or about 3 months or more months, or about one or more years, and any range derivable therein, prior to and/or after administering the AR inhibitor.

Various combinations of a AR inhibitor(s) and a second therapeutic may be employed in the present invention, where a AR inhibitor is “A” and the secondary agent, such as a antibiotic or other anti-inflammatory treatment, is “B”:

| | | | | | | |
|---|---|---|---|---|---|---|
| A/B/A | B/A/B | B/B/A | A/A/B | A/B/B | B/A/A | B/B/B/A |
| B/B/A/B | A/A/B/B | A/B/A/B | A/B/B/A | B/B/A/A | B/A/B/A | B/A/A/B |
| A/A/A/B | B/A/A/A | A/B/A/A | A/A/B/A | A/B/B/B | B/A/B/B | |

Administration of modulators to a cell, tissue or organism may follow general protocols for the administration of agents for the treatment of inflammatory bowel disease, macular degeneration, or posterior capsule opacification. It is expected that the treatment cycles would be repeated as necessary. In particular embodiments, it is contemplated that various additional agents may be applied in any combination with the present invention. Agents include antibiotics (for gram-positive and gram negative bacteria), anti-inflammatory drugs, and immunosuppressant drugs, which are well known to those of skill in the art and frequently commercially available.

In such combinations, AR inhibitors and other active agents may be administered together or separately. In addition, the administration of one agent may be prior to, concurrent to, or subsequent to the administration of other agent(s).

### VII. EXAMPLES

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

### Material and Methods

Materials

Dulbecco's modified Eagle's medium (DMEM) with 4 mM L-glutamine and 1 g/l glucose, phosphate-buffered saline (PBS), gentamicin solution, 0.25% trypsin/EDTA solution, and fetal bovine serum (FBS) were purchased from Invitrogen-Gibco (Grand Island, N.Y.). Antibodies against β-crystallin, filensine, ICAM-1 and glyceraldehyde phosphate dehydrogenase (GAPDH) were from Santa Cruz Biotechnology (Santa Cruz, Calif.); antibodies against α-SMA from research diagnostic Inc (Concord, Mass.) and antibodies against Proliferating cell nuclear antigen (PCNA), phospho-p38, phospho-ERK1/2 and phospho-SAPK/JNK and total-p38, -ERK1/2 and -SAPK/JNK were from Cell Signaling Inc. (Beverly, Mass.). AR inhibitors Sorbinil and Zopolrestat were gift from Pfizer (Groton, Conn.) and Fidarestat was from Sanwa Kagaku Kenkyusho Co. Ltd, Japan. The transfection reagent (LipofectAMINE Plus) and reduced-serum cell-culturing medium (OptiMEM) were obtained from Invitrogen-Life Technologies (Gaithersburg, Md.). Consensus oligonucleotides for NF-κB (5′-AGTTGAGGGGACTTTCCCAGGC-3′ (SEQ ID NO:1)) transcription factor was from Promega Corp. (Madison, Wis.); Basic-fibroblast growth factor (b-FGF), 3-(4,5-dimethylthiazol-2-yl)-2,5-diphenyl tetrazolium bromide (MTT) and other reagents used in the electrophoretic mobility shift-gel assay (EMSA) and Western blot analysis were obtained from Sigma-Aldrich (St. Louis, Mo.). All other reagents used were of analytical grade.

Explant Culture of Pig Eye Capsular Bags

Pig eye capsular bags were prepared by phacoemulsification followed by capsulorhexis and incubated in Dulbecco's Modified Eagle's Medium (DMEM) supplemented with 10% FBS and 1% penicillin and streptomycin without or with AR inhibitors, sorbinil (50 μM) and fidarestat (10-30 μM) for 7 days in 5% CO₂ condition at 37° C. Medium was replaced with fresh medium without or with AR inhibitors every day. At the end of incubation, capsular bags were examined under the dissection microscope, cut into four pieces and flat mounted on glass slide with cellular side facing up. The pieces of capsular bags were fixed with 4% paraformaldehyde for 3-4 h at 4° C. After fixation, they were washed, transferred to 70% alcohol and stored at 4° C. until used for immunohistochemical analysis.

Immunohistochemical Analysis of Pig Eye Capsular Bag Specimens

The capsular bag specimens flat mounted on slides were washed thrice with PBS and incubated with blocking solution containing 2% BSA, 0.1% Triton-X100, 5% normal goat serum for 1 h at room temperature. Subsequently specimens were washed, incubated with antibodies against α-SMA, β-crystallin, PCNA, and ICAM-1 for 1 h at room temperature. The slides were stained with either LSAB-HRP system (Dako, Carpinteria, Calif.) or incubated with respective FITC labeled secondary antibodies. The samples were examined by using Nikon epifluoroscence microscope (EPI-800 microscope) connected to a computer with Metamorph software.

Determination of Lens Epithelial Cell Growth in Pig Eye Capsular Bags

The pig eye capsular bags were incubated for 7 days as described above. The lens epithelial cells were detached from capsular bags by incubating with 0.25% trypsin-EDTA solution. The trypsinized cells were harvested, washed with PBS and re-suspended in PBS. To determine cell growth, equal volumes of cell suspension and trypan blue were mixed and the cells which excluded trypan blue dye were counted manually by using a hemocytometer.

Tissue Culture of HLEC

Adenovirus SV-40 viral DNA-transformed human lens epithelial cells (HLE-B3; HLEC) were purchased from ATCC. Cells were maintained in DMEM supplemented with 20% heat-inactivated FBS, and 50 μg/ml gentamicin. The cells were grown in a humidified incubator at 37° C. and 5% CO₂. The HLEC were pretreated with 20 μM of AR inhibitors sorbinil or zopolrestat for overnight in medium containing 0.5% FBS followed by stimulation with various concentrations (0-100 ng/mL) of b-FGF for 24 h and cell viability and expression of α-SMA, β-crystallin and ICAM-1 was quantified. To investigate the effect of b-FGF on NF-κB and MAPK activation, HLEC were starved in medium containing 0.5% FBS with or without AR inhibitors for overnight. The cells were washed with serum-free medium followed by incubation in serum-free medium without or with AR inhibitor containing 50 ng/mL b-FGF for different time intervals.

Cell Viability Assays

The HLEC were grown to confluence, harvested and plated at 5000 cells/well in 96-well plates. The cells were growth-arrested by incubation in 0.5% FBS medium without or with AR inhibitor (20 μM) for 24 h. After 24 h, the cells were stimulated with b-FGF (0, 10, 20, 50, and 100 ng/mL), and incubated for an additional 24 h. Cell viability was determined by MTT assay as described by us earlier (Ramana et al., 2003). Briefly, after incubation of HLEC, 10 μL of 5 mg/mL MTT in PBS were added to each well and incubated further for 2 h at 37° C. The medium was removed and formazan granules obtained were dissolved in 100% dimethyl sulfoxide (DMSO), and absorbance at 562 nm was detected with an ELISA plate reader. The cell viability was also determined by cell counting with a hemocytometer as described above.

Western Blot Analysis

HLEC incubated under various conditions described above were washed twice with ice-cold PBS and lysed in ice-cold lysis buffer containing 50 mM HEPES [pH 7.6], 10 mM KCl, 0.5% NP-40, 1 mM DTT, 1 mM phenylmethylsulfonylfluoride (PMSF), and 1:100 dilution of protease inhibitor cocktail (Sigma, Saint Louise, Mo.) for 15 min at 4° C. The crude lysates were cleared by centrifugation at 12,000 g for 10 min at 4° C. Aliquots of the lysates containing equal amount of protein (40 μg) were separated on 10% SDS-polyacrylamide gels and transferred to polyvinylidene difluoride membranes (Immobilon; Millipore, Bedford, Mass.). The membranes were then incubated in blocking solution containing 5% wt/vol dried fat-free milk and 0.1% vol/vol Tween-20 in tris-buffered saline. Subsequently, the membranes were incubated with antibodies against A-SMA, β-crystallin, ICAM-1, phospho-p38, phospho-ERK1/2, phospho-SAPK/JNK, and total-p38, -ERK1/2 and -SAPK/JNK and GAPDH. The membranes were washed and probed with the respective HRP-conjugated secondary antibodies (SouthernBiotech, Birmingham, Ala.) and visualized by chemiluminescence (Pierce biotechnology, Rockford, Ill.).

Electrophoretic Mobility Shift Assay (EMSA)

The HLEC in 150 cm² culture flasks were pretreated without or with AR inhibitors for 24 h in serum-free medium, followed by treatment with b-FGF (50 ng/ml) for 1 h at 37° C. The nuclear extracts were prepared as described earlier (Ramana et al., 2003). The Consensus oligonucleotides for NF-κB transcription factors were 5′-end labeled using T4 polynucleotide kinase. EMSA was performed as described earlier (Ramana et al., 2003.). The specificity of the assay was examined by competition with an excess of unlabeled oligonucleotide and supershift assays with antibodies to p65.

NF-κB-Dependent Secretory Alkaline Phosphatase (SEAP) Expression Assay

To examine NF-κB promoter activity in HLEC in response to b-FGF treatment, cells (1×10⁵ cells/well) were plated in 24-well plate. The cells were starved for 16 h in 0.5% FBS medium without or with AR inhibitors and transfected with pNF-κB-SEAP2-construct and pTAL-SEAP control plasmid (Clontech, USA) using Lipofectamine plus (Invitrogen, Carlsbad, Calif.) transfection reagent following suppliers instructions. After 6 h of transfection, cells were stimulated with b-FGF (50 ng/ml) for 48 h. The cell culture media were harvested and centrifuged at 5000 rpm and supernatants were stored at −80° C. The medium was thawed and used for chemiluminescent secretory alkaline phosphatase (SEAP) assay using Great EscAPe™ SEAP reporter assay system according to protocol essentially as described by the manufacturer, (BD Biosciences, Palo Alto, Calif.) using a 96-well chemiluminescence plate reader. All the suggested controls by manufacturers were used in the assay.

RNA Interference Ablation of AR

HLEC were grown to 60% confluence in DMEM supplemented with 20% FBS in 6-well plate. The cells were incubated with OptiMEM medium containing the AR-siRNA (AATCGGTGTCTCCAACTTCAA (SEQ ID NO:2)) or scrambled siRNA (AAAATCTCCCTAAAT CATACA (SEQ ID NO:3); control) to a final concentration of 100 nM and the RNAiFect™ transfection reagent (Qiagen) as described by the inventors earlier (Ramana et al., 2004). Briefly, for each well, 2 μg AR siRNA was diluted in serum-free medium to give a final volume of 100 μl and incubated with 6 μl RNAiFect® for 15 min at room temperature. The transfection mixture was added to the respective wells, each containing 1900 μl complete medium (20% fetal bovine serum), and incubated for 24 h. After 24 h, the medium was replaced with fresh DMEM (serum free) for another 24 h before stimulation with b-FGF. Changes in the expression of AR were estimated by Western blot analysis using anti-AR antibodies.

Immunocytochemical Analysis

For immunocytochemical analysis, HLEC were plated in chambered slides and grown to ˜70% confluence. The cells were growth arrested by incubating in 0.5% FBS medium for 24 h without or with AR inhibitors. After treatment with b-FGF for 24 h, the cells were washed and quickly fixed in methanol (pre-chilled at −20° C.) for 10 min. The slides were air dried and washed three times with PBS and blocked in the blocking solution containing 2% BSA, 0.1% Triton-X100, and 5% normal goat serum for 1 h at room temperature. Slides were washed twice and incubated with antibodies against A-SMA, β-crystallin, filensin, and ICAM-1 for 1 h at room temperature. Subsequently, the slides were washed thrice with PBS and stained using FITC labeled secondary antibodies, washed and mounted using flour-save mounting medium with DAPI (Vector laboratories Inc, Burlingame, Calif.). The sections were examined under Nikon epifluoroscence microscopy (EPI-800 microscope).

Statistical Analysis

Data presented as mean±SD and p values were determined by unpaired Student's t test using Microsoft Office Excel 2003 software. p<0.05 was considered as statistically significant.

### Example 2

### AR Inhibition Prevents Epithelial Cell Growth in Pig Eye Capsular Bags

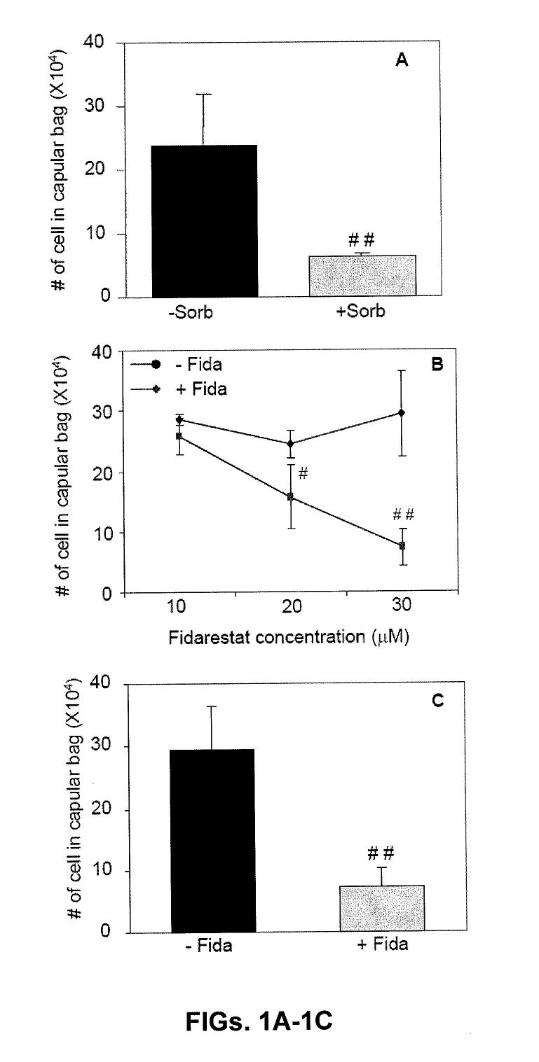

Phacoemulsification technique for cataract surgery in humans was used to operate enucleated pig eyes. After removing the lens, a capsular tension ring was inserted in the capsular bag. The capsular bag was then cultured in complete growth-medium without or with AR inhibitors. The lens epithelial cells first appeared after 2-3 days in culture near the equator then migrated towards anterior as well as posterior regions of the capsule covering it entirely in 7 days and showed distinct folding in the central region. After 7 days in culture, the ring was removed and the cells were harvested by trypsinization. The harvested cells were counted using trypan blue exclusion principle. As shown in FIG. 1A, lens epithelial cells showed massive growth in the complete medium. Epithelial cells counted from three capsular bags in three subsequent batches of cultures showed a significant (˜74%) inhibition of cell growth by sorbinil (FIG. 1A). Similar results were obtained with another structurally distinct AR inhibitor, fidarestat, which showed a dose-dependent response in inhibiting the growth of LEC in pig eye capsular bags (FIG. 1B). The most effective dose at which fidarestat significantly (p<0.001) inhibited the LEC growth was 30 μM (FIG. 1B).

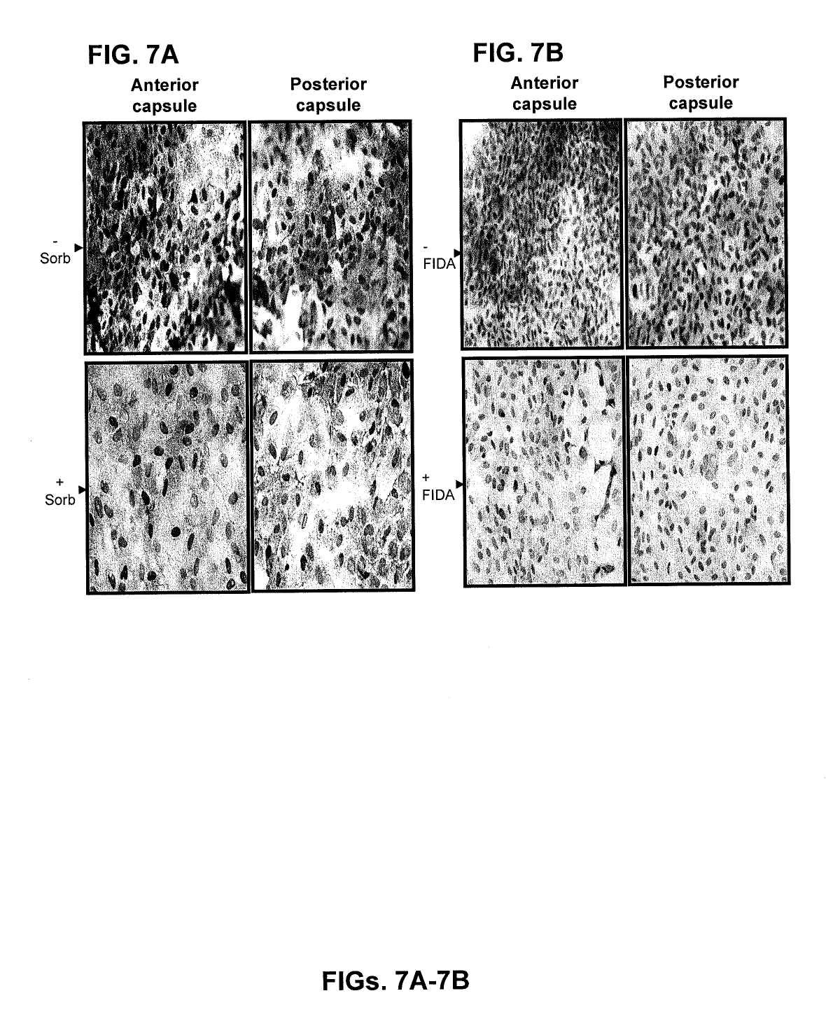

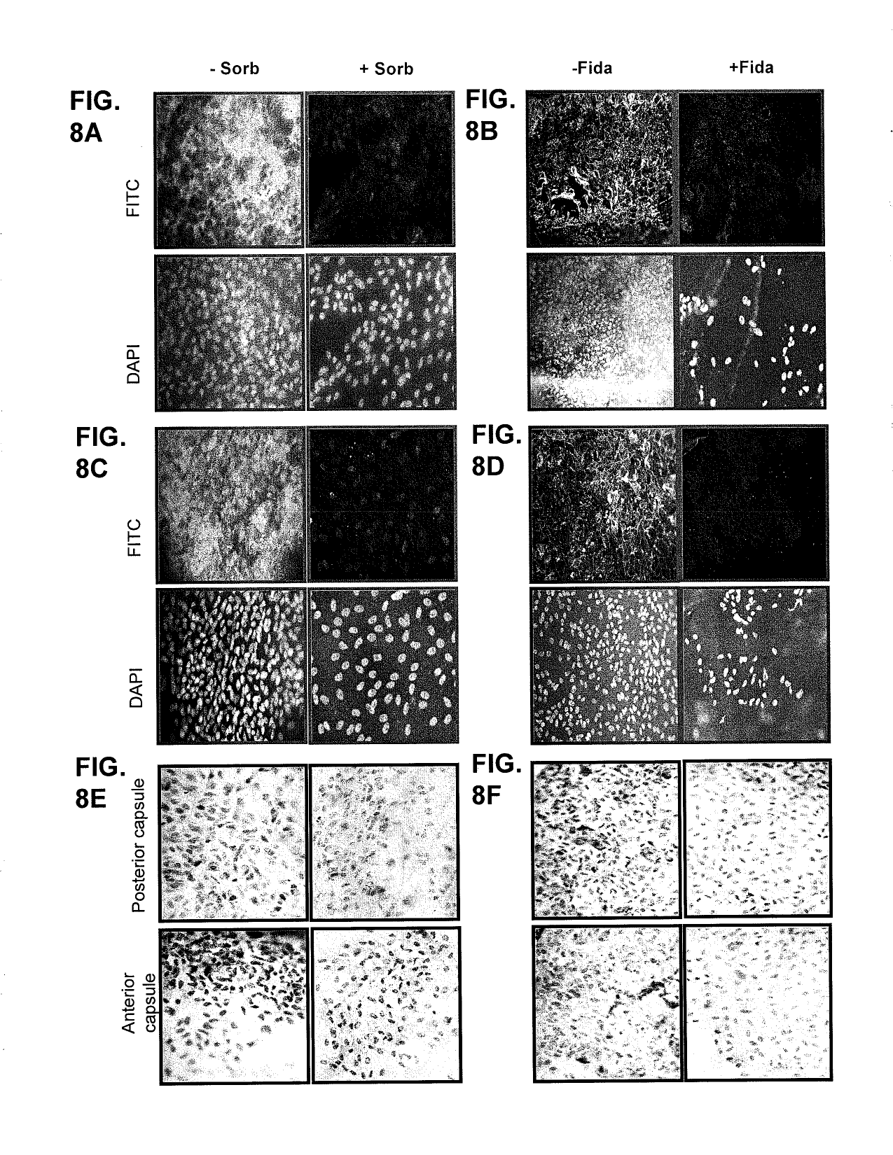

Subsequent to observation that AR inhibition could block the cell growth, the inventors examined the status of the residual cells i.e. whether they were still in cell division mode. This was examined by fixing the capsular bag in 4% paraformaldehyde and immunostaining with anti-PCNA antibodies. As shown in FIGS. 7A-B, in capsular bags cultured without AR inhibitor, a large number of cells were PCNA positive indicating that they were still dividing, whereas in AR inhibitor-treated capsular bags, the number of PCNA positive cells were significantly low, indicating non-proliferating nuclei. Subsequently, the ratio of PCNA positive cells with that of total cells in both groups was quantified. More than 70% cells were PCNA positive in control while in AR inhibitor-treated capsular bags only approximately 15% cells were PCNA positive. These results show that AR inhibition could block the growth/proliferation of residual cells after phacoemulsification of lens.

### Example 3

### AR Inhibition Prevents Trans-Differentiation of LEC in Pig Eye Capsular Bags

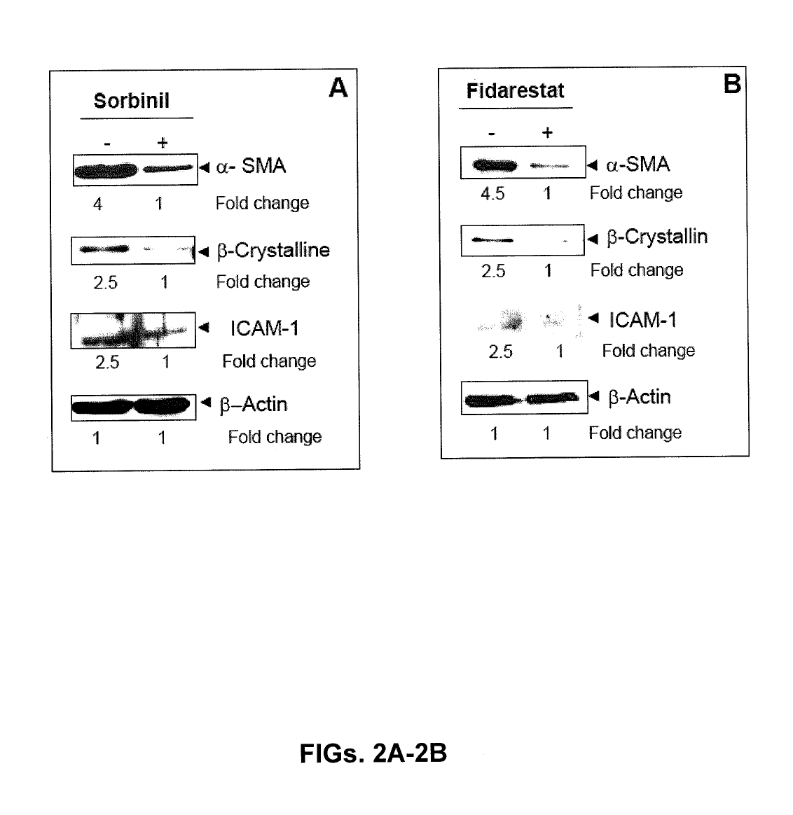

The inventors next examined the effect of AR inhibition on the trans-differentiation of LEC in pig eye capsular bags. As shown in FIGS. 2A-B, lens epithelial cells from pig eye capsular bags cultured without AR inhibitors showed 3-5 fold increased expression of A-SMA and β-crystallin compared to those cultured with AR inhibitors. Similarly the expression of ICAM-1 was increased by ˜2 fold after 7-days culture and both AR inhibitors were able to prevent it.

Immunohistochemical studies on flat-mounted capsular bags after 7 days of culture showed that a large number of cells in control capsular bags expressed α-SMA and β-crystallin in the absence of AR inhibitor, sorbinil (FIGS. 8A and 8C), where as in AR inhibitor-treated capsular bags, only few cells expressed α-SMA and β-crystallin. Treatment with fidarestat also showed similar inhibition in the expression of α-SMA and β-crystallin (FIGS. 8B and 8D). Nishi et al. (1997.) have suggested that cell adhesion molecules such as ICAM-1 could be involved in adhesion of LEC to extra-cellular matrix and play a crucial role in cell growth and migration. Therefore, the inventors examined the expression of ICAM-1 in the pig eye capsular bag and found that in control capsular bags the epithelial cells expressed ICAM-1 protein at both anterior and posterior capsule. In AR inhibitors-treated capsular bags, the expression of ICAM-1 was markedly inhibited (FIGS. 8E-F). These results thus indicate that AR inhibition could be successfully used to block the trans-differentiation of epithelial cells subsequent to phacoemulsification of lens in cataract surgery which could prevent PCO.

### Example 4

### AR Inhibition Prevents b-FGF-Induced HLEC Growth

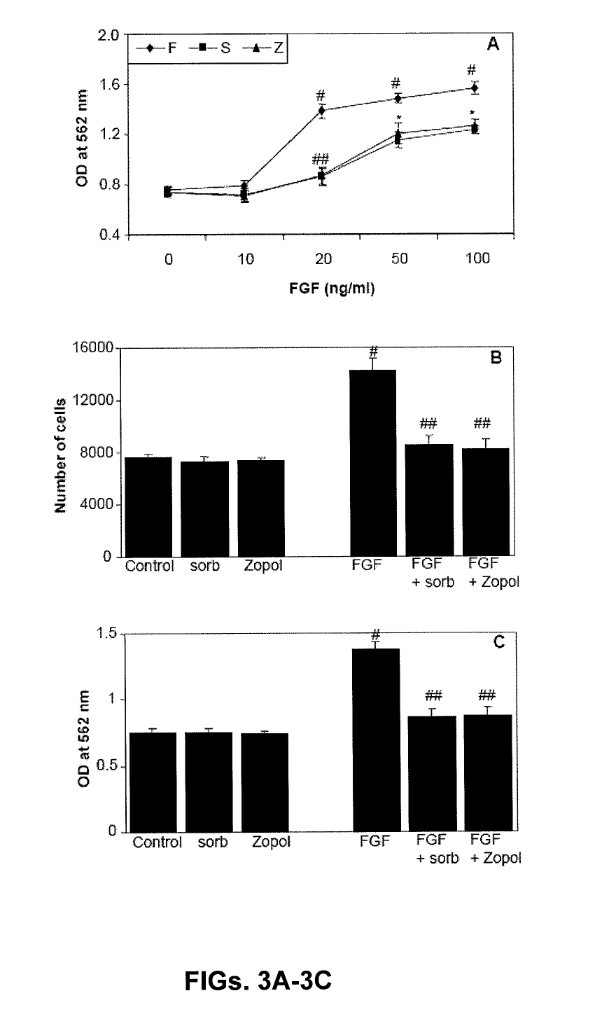

In order to confirm their findings in the pig eye capsular bag model and to understand the molecular mechanisms by which AR inhibition prevents lens epithelial cell proliferation, the inventors examined the effect of AR inhibitors on b-FGF-induced proliferation of cultured HLEC. As shown in FIG. 3A, at lower concentrations of b-FGF (<20 ng/ml) the cells proliferated vigorously, whereas at higher concentration (>50 ng b-FGF/ml) cell proliferation attained a plateau and cells started undergoing differentiation and/or transdifferentiation. Inhibition of AR by two structurally distinct inhibitors significantly (p<0.01) blocked the cell growth induced by b-FGF as determined by cell counting and MTT assay (FIGS. 3B-C).

### Example 5

### AR Inhibition Prevents Expression of α-SMA, β-Crystallin and ICAM-1 in HLEC

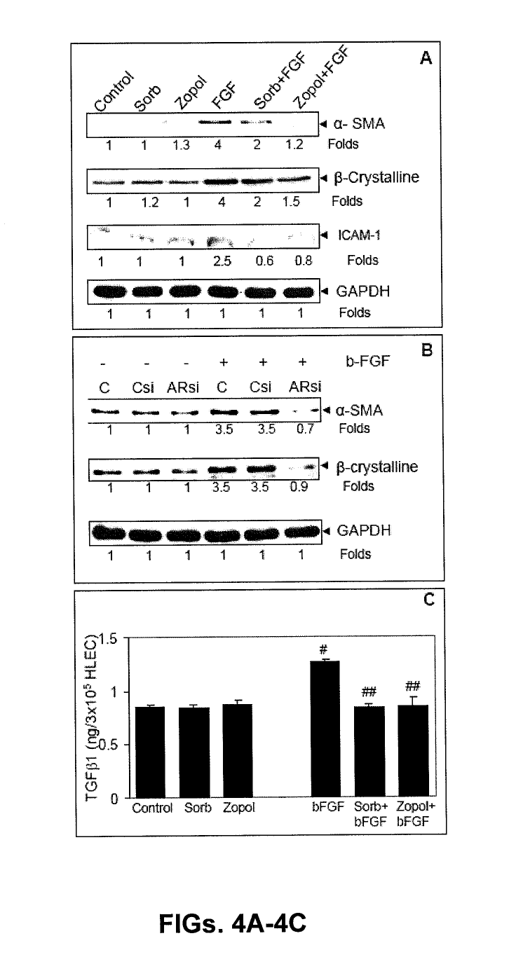

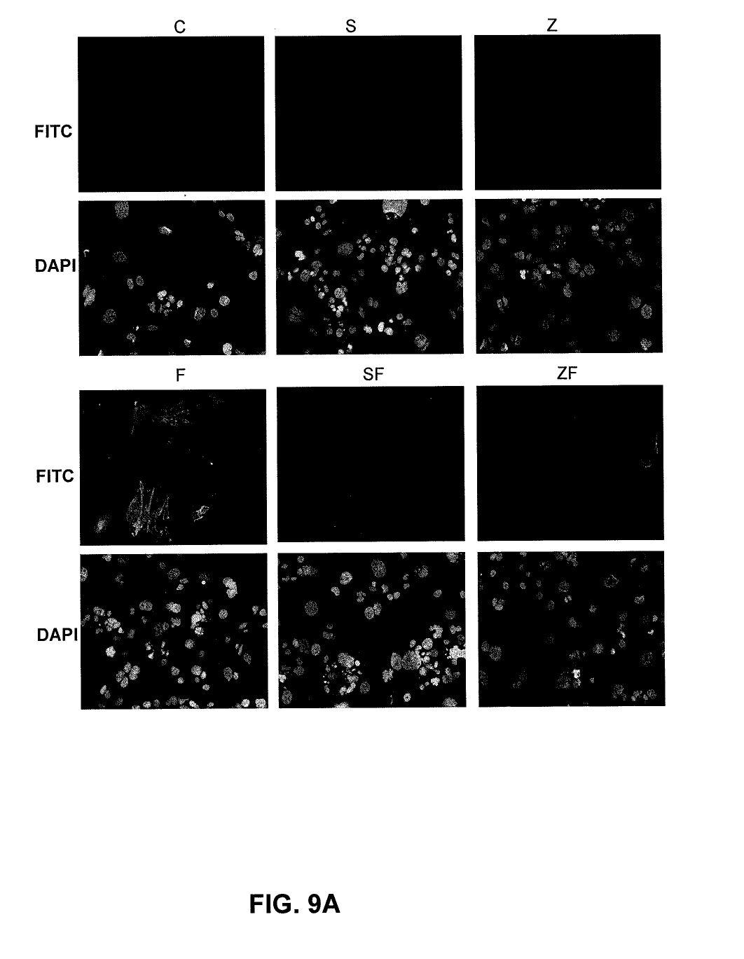

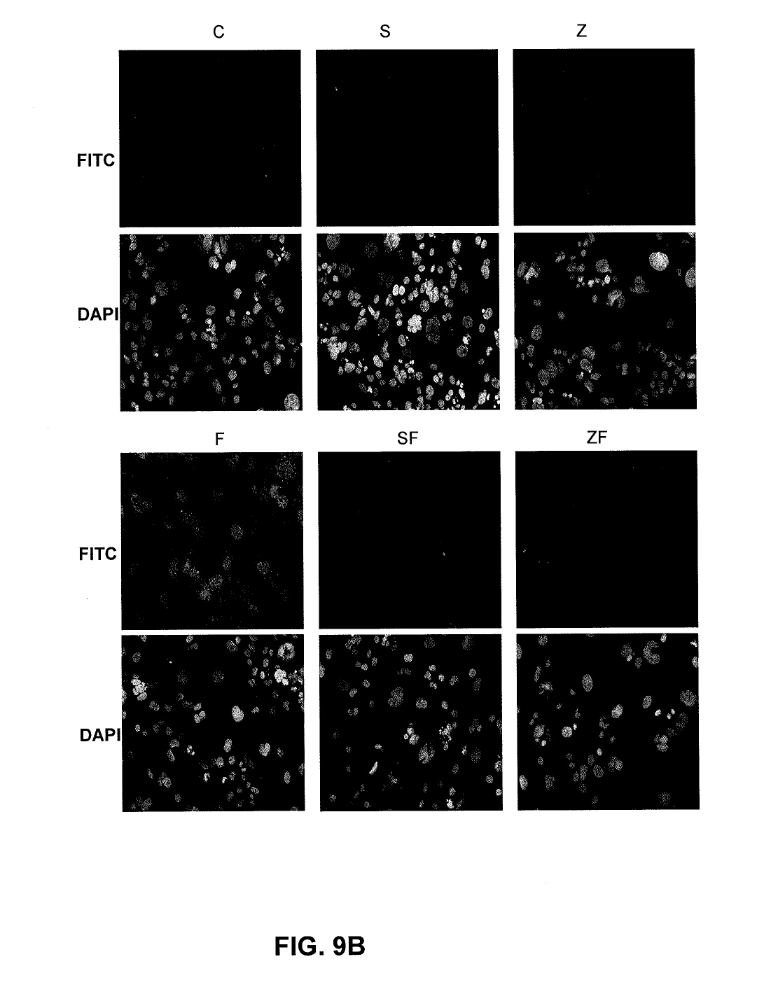

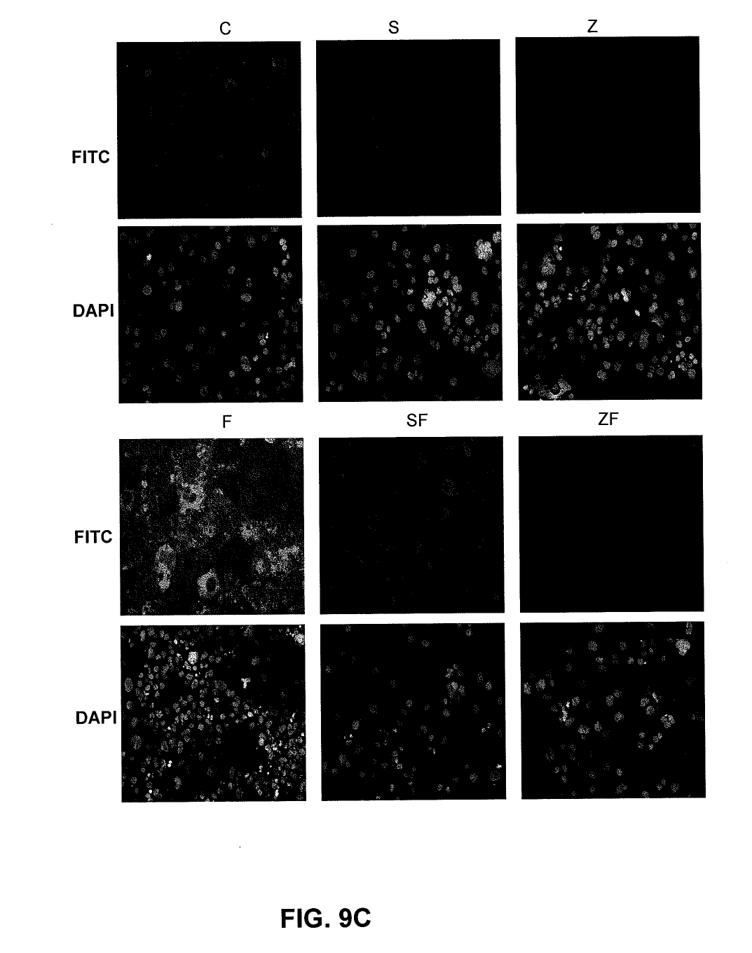

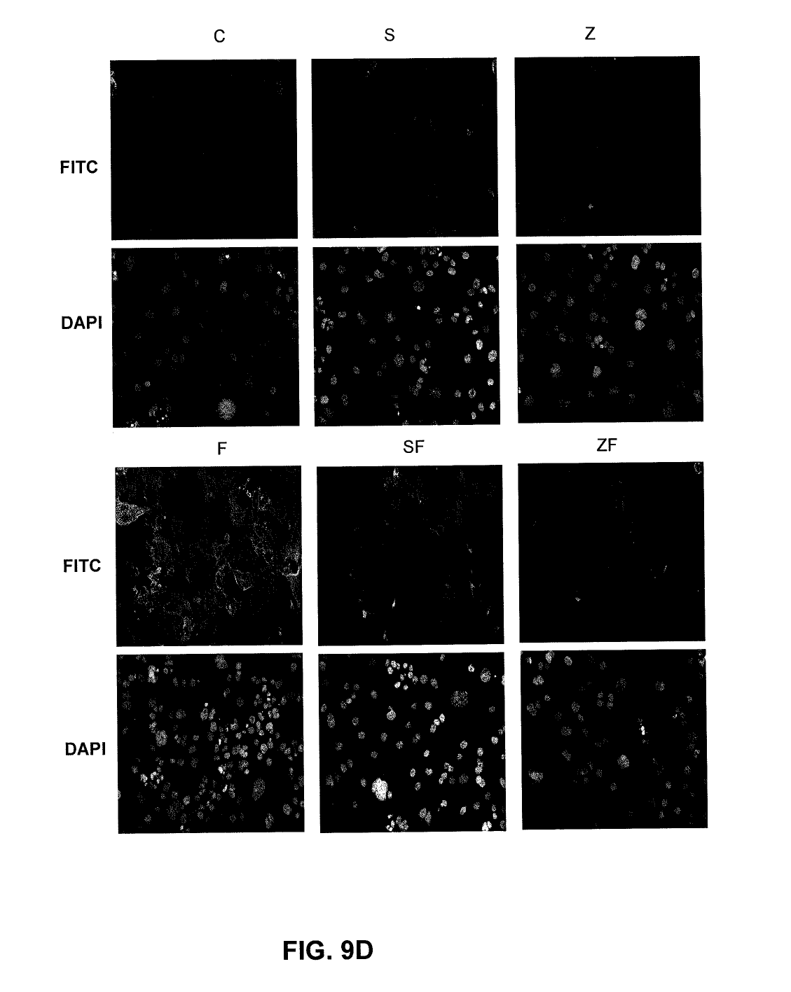

Since it is already known that b-FGF at higher doses induces the expression of differentiation markers (Lovicu et al., 2001; Iyengar et al., 2007.), the inventors next examined the effect of AR inhibition on b-FGF-induced expression of β-crystallin and α-SMA in HLEC. As shown in FIG. 4A, treatment of HLEC with 50 ng/ml b-FGF caused ˜4-fold increase in the expression of β-crystallin, and α-SMA and ˜2.5-fold increase in the expression of adhesion molecule ICAM-1 and AR inhibitors significantly (˜90%) inhibited the increase (FIG. 4A). However, treatment with AR inhibitors alone did not cause any significant change in the expression of these proteins. Although sorbinil and zopolrestat are specific inhibitors of AR, to rule out any non-specific effect of pharmacological inhibitors, the inventors ablated AR message by transfecting the HLEC with AR-siRNA. As shown in FIG. 4B, b-FGF-induced increase in the expression of A-SMA and β-crystallin in untransfected (C) and scrambled siRNA oligo (Csi) transfected HLEC but not in the AR-siRNA transfected cells suggesting that AR plays a crucial role in the aberrant expression of α-SMA and β-crystallin in HLEC. Further, immuno-cytochemical analysis also demonstrates that AR inhibition significantly blocked b-FGF-induced expression of A-SMA, β-crystallin, filensin and ICAM-1 in HLEC (FIGS. 9A-D).

### Example 6

### AR Inhibition Prevents b-FGF-Induced Activation of NF-KB in HLEC

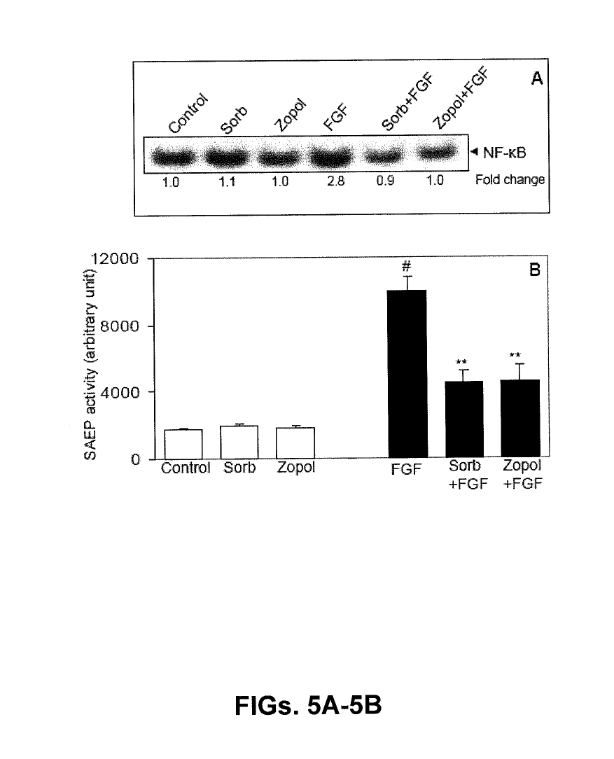

Since activation of NF-κB mediates expression of various marker proteins and cytokines, the inventors next examined the effect of AR inhibition on b-FGF-induced NF-κB activation in HLEC. Treatment with b-FGF caused 3-fold increase in the activation of NF-κB as indicated by DNA binding capacity determined by EMSA (FIG. 5A). Inhibition of AR by sorbinil or zopolrestat significantly (>90%) prevented the b-FGF-induced NF-κB DNA binding capacity. However, sorbinil or zopolrestat alone did not affect the basal activity of NF-κB. The inventors further confirmed their results with more sensitive NF-κB-dependent reporter assay. As shown in FIG. 5B, b-FGF caused >5-fold activation in the NF-κB dependent reporter SEAP activity which was significantly (˜60%; p<0.01) inhibited by AR inhibitors. These results suggest that inhibition of AR could modulate b-FGF-induced activation of redox-sensitive transcription factors, which could be responsible for preventing the growth and trans-differentiation of LEC and thereby prevent PCO.

### Example 7

### AR Inhibition Prevents b-FGF-Induced Phosphorylation of MAPKs

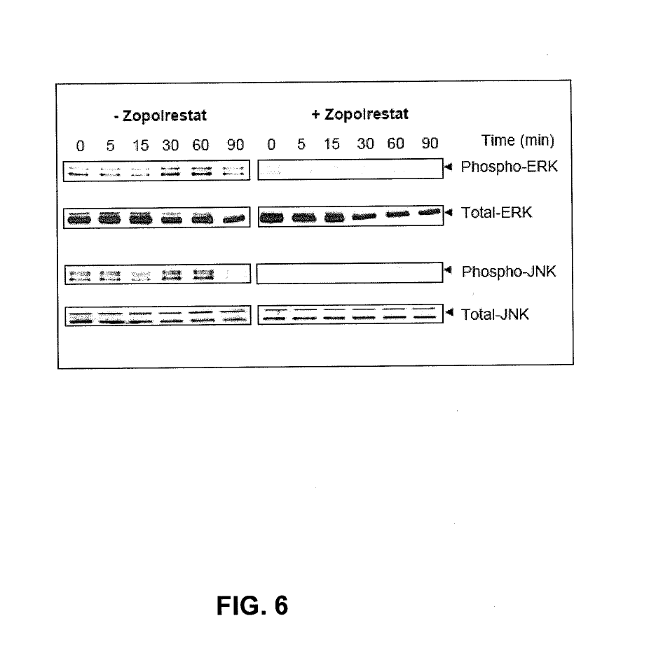

MAPKs are known to regulate the phosphorylation and activation of NF-κB; therefore, the inventors next examined the effect of AR inhibition on b-FGF-induced activation of MAPKs. As shown in FIG. 6, treatment with b-FGF enhanced phosphorylation of ERK1/2-MAPK, and SAPK/JNK in HLEC and pre-treatment with AR inhibitor prevented it. There was no change in the expression of total ERK1/2 and SAPK/JNK proteins. The inventors did not observe any change in phosphorylation of p38-MAPK in response to b-FGF treatment, suggesting that MAPK and SAPK/JNK are the main mediators of growth factor-induced NF-κB activation in HLEC.

### REFERENCES

The following references, to the extent that they provide exemplary procedural or other details supplementary to those set forth herein, are specifically incorporated herein by reference.
U.S. Pat. No. 4,130,714
U.S. Pat. No. 4,251,528
U.S. Pat. No. 4,436,745
U.S. Pat. No. 4,438,272
U.S. Pat. No. 4,464,382
U.S. Pat. No. 4,540,704
U.S. Pat. No. 4,600,724
U.S. Pat. No. 4,734,419
U.S. Pat. No. 4,771,050
U.S. Pat. No. 4,791,126
U.S. Pat. No. 4,831,045
U.S. Pat. No. 4,883,410
U.S. Pat. No. 4,883,800
U.S. Pat. No. 4,980,357
U.S. Pat. No. 5,037,831
U.S. Pat. No. 5,066,659
U.S. Pat. No. 5,252,572
U.S. Pat. No. 5,270,342
U.S. Pat. No. 5,354,855
U.S. Pat. No. 5,430,060
U.S. Pat. No. 5,447,946
U.S. Pat. No. 5,582,981
U.S. Pat. No. 5,756,291
U.S. Pat. No. 5,780,610
U.S. Pat. No. 5,792,613
U.S. Pat. No. 5,840,867
U.S. Pat. No. 5,990,111
U.S. Pat. No. 6,127,367
U.S. Pat. No. 6,380,200
U.S. Pat. No. 6,696,407
U.S. Pat. No. 6,720,348
U.S. patent application Ser. No. 10/462,223
U.S. patent application Ser. No. 11/210,283
U.S. Patent Publication 20030105051
U.S. Patent Publication 20030059944
U.S. Provisional Application 60/388,213
U.S. Provisional Application 60/603,725
Apple et al., Surv. Opthalmol. 1992; 37:73-116, 1992.
Barany and Merrifield, Anal Biochem. 95(1):160-70, 1979.
Barisani et al., FEBS Lett. 469(2-3):208-12, 2000.
Berzal-Herranz et al., Genes Dev. 6(1):129-34, 1992.
Bhatnager and Srivastava, Biochem Med Metab Biol. 48(2):91-121, 1992.
Biswas et al., Ophthalmic Res. 31:140-142, 1999.
Brummelkamp et al., Science, 296(5567):550-3, 2002.
Burgstaller et al., Curr Opin Drug Discov Devel. 5(5):690-700, 2002.
Cech et al., Cell. 27(3 Pt 2):487-96, 1981.
Chen et al., Mol. Vis. 13:374-387, 2007.
Cheung et al., Diabetes 54:3119-3125 2005.
Choi et al., Invest Opthalmol Vis Sci. 2004; 45:2696-2704, 2004.
Chowrira et al., J Biol. Chem. 268(26):19458-62, 1993.
Chowrira, et al., J Biol. Chem. 269(41):25856-64, 1994.
Cortina et al., Graefes Arch. Clin. Exp. Opthalmol. 235:180-185, 1997.
De Iongh et al., Cells Tissues Organs. 179:43-55, 2005
Dixit et al., J Biol. Chem. 275:21587-21595, 2000.
Doganay et al., J. Cataract. Refract. Surg. 28:1457-1462, 2002.
Elbashir et al., Genes Dev. 15(2):188-200, 2001.
Forster and Symons, Cell. 50(1):9-16, 1987.
Hales et al., Invest Opthalmol Vis Sci. 36:1709-1713, 1995.
Haseloff and Gerlach, Nature. 334(6183):585-91, 1988.
Hepsen et al., J. Cataract. Refract. Surg. 23:1572-1576, 1997.
Humber, “Aldose Reductase Inhibition: An Approach to the Prevention of Diabetes Complications”, Porte, ed., Ch. 5, pp. 325-353.
Inan et al., Graefes Arch. Clin. Exp. Opthalmol. 239:693-697, 2001.
Iyengar et al., Differentiation 75:662-668, 2007.
Jiang et al., J. Ocul. Pharmacol. Ther. 22:93-102, 2006.
Joyce, Gene. 82(1):83-7, 1989.
Kim and Cech, Proc Natl Acad Sci USA. 84(24):8788-92, 1987.
Lang and McAvoy, In: Development of the Ocular Lens, Lovicu and Robinson (Eds.), New York, Cambridge University Press, 261-289, 2004.
Lee and Chung, FASEB J. 13:23-30, 1999.
Lee and Joo, Invest Opthalmol Vis Sci. 40:2025-2032, 1999.
Lieber and Strauss, Mol Cell Biol. 15(1):540-51, 1995.
Liu et al., Invest. Opthalmol. Vis. Sci. 35:388-401, 1994.
Lovicu and McAvoy, Development 128:5075-5084, 2001.
Malone, Diabetes. (11):861-4, 1980.
Mansfield et al., Mol. Vis. 10:728-737, 2004.
Meacock et al., Br. J. Opthalmol. 84:332-336, 2000.
Merrifield, Science. 232(4748):341-7, 1986.
Michel and Westhof, J Mol. Biol. 216(3):585-610, 1990.
Nakamura et al., Free Radic Biol Med. 29(1):17-25, 2000.
Nishi et al., Invest Opthalmol V is Sci. 1997; 38:579-585, 1997.
Nishi, J. Cataract Refract. Surg. 25:106-117, 1999.
O'Connor et al., Biochem J 343 Pt 2:487-504, 1999.
Ohadi et al., Curr. Opin. Opthalmol. 2:46-52, 1991.
Perriman et al., Gene. 113(2):157-63, 1992.
Perrotta and Been, Biochemistry. 31(1):16-21, 1992.
Pladzyk et al., Invest Opthamol V is Sci. 47:5395-403, 2006.
Prieto et al., Ann Allergy Asthma Immunol. 97:175-181, 2006.
Prody et al., Science. 231(4745):1577-1580, 1986.
Ramana et al., Biochemistry. 39:12172-12180, 2000.
Ramana et al., Chem. Biol. Interact. 143-144:587-596, 2003.
Ramana et al., Diabetes. 53:2910-2920, 2004.
Ramana et al., FASEB J 17:315-317, 2003.
Ramana et al., FASEB J 18:1209-1218, 2004.
Ramana et al., J Biol. Chem. 275:32063-32070, 2002.
Ramana et al., J Biol. Chem. 28:33019-33029, 2006.
Reinhold-Hurek and Shub, Nature. 357(6374):173-76, 1992
Remington's Pharmaceutical Sciences, 18th Ed. Mack Printing Company, 1990
Rittner et al., J Clin Invest. 103(7):1007-13, 1999.
Ruef et al., Arterioscler Thromb Vasc Biol. 20(7): 1745-52, 2000.
Sarver et al., Science. 247(4947):1222-5, 1990.
Scanlon et al., Proc Natl Acad Sci USA. 88(23):10591-5, 1991.
Seki et al., Folia Opthalmology Japan 43:1452-1456, 1992.
Seo et al., J Biol. Chem. 275(39):30355-62, 2000.
Sheetz et al., JAMA 288:2579-2588, 2002.
Shinmura et al., Proc Natl Acad Sci USA. 97(18):10197-202, 2000.
Sioud et al., J Mol. Biol. 223(4):831-5, 1992.
Spycher et al., FASEB J. 11(2):181-8, 1997.
Srivastava et al., Adv Exp Med. Biol. 463:501-7, 1999.
Srivastava et al., Biochem Biophys Res Commun. 217:741-746, 1995.
Srivastava et al., Biochemistry. 37(37):12909-17, 1998.
Srivastava et al., Endocr Rev. 26:380-392, 2005.
Sui et al., Proc Natl Acad Sci USA. 99(8):5515-20, 2002.
Symonds et al., Exp. Eye Res. 82:693-699, 2006.
Symons, Annu Rev Biochem. 61:641-71, 1992.
Symons, Nucleic Acids Res. 9(23):6527-37, 1981.
Tam et al., Int J Pept Protein Res. 21(1):57-65, 1983.
Tammali et al., Cancer Res. 66:9705-9713, 2006.
Thompson, et al., Nat. Med. 1(3):277-8, 1995.
Tomlinson et al. Pharmac. Ther. 54:151-194, 1992.
Vander Jagt et al., Biochim Biophys Acta. 1249(2):117-26, 1995.
WO 03/012052
Wormstone et al., Invest. Opthalmol. Vis. Sci. 41:4216-4222, 2000.
Yabe-Nishimura, Pharmacol Rev. 50(1):21-33, 1998.
Yadav et al., Invest Opthalmol Vis Sci. 48:4634-4642, 2007.
Yang et al., Circulation. 102(25):3046-52, 2000.
Yuan and Altman, Science. 263(5151):1269-73, 1994.
Yuan et al., Proc Natl Acad Sci USA 89(17):8006-10, 1992.
Zenon et al., Clin. Pharm. 9:446-457, 1990.

1. A method of treating posterior capsule opacification in a subject comprising administering to a subject diagnosed with or at risk of developing inflammatory bowel disease, macular degeneration, or posterior capsule opacification an amount of a pharmaceutically acceptable composition comprising an aldose reductase inhibitor sufficient to prevent, ameliorate, or attenuate posterior capsule opacification.

2. The method of claim 1, wherein the composition is administered one or more times.

3. The method of claim 1, wherein the aldose reductase inhibitor is administered to the patient as a prodrug.

4. The method of claim 1, wherein the patient is administered the composition directly, locally, topically, orally, endoscopically, intratracheally, intratumorally, intravenously, intralesionally, intramuscularly, intraperitoneally, regionally, percutaneously, or subcutaneously.

5. The method of claim 4, wherein the aldose reductase inhibitor is administered orally.

6. The method of claim 1, wherein the aldose reductase inhibitor is a small molecule or an inhibitory RNA.

7. The method of claim 6, wherein the aldose reductase inhibitor is an siRNA, an inhibitory nucleic acid, a carboxylic acid, a hydantoins, a pyridazinone, or a pharmaceutically acceptable derivative thereof.

8. The method of claim 7, wherein the aldose reductase inhibitor is fidarestat, sorbinil, epalrestat, ponalrestat, methosorbinil, risarestat, imirestat, ALO-1567, quercetin, zopolrestat, AD-5467, NZ-314, M-16209, minalrestat, AS-3201, WP-921, luteolin, tolrestat, EBPC, fidarestat, or a pharmaceutically acceptable derivative thereof.

9. The method of claim 8, wherein the aldose reductase inhibitor is fidarestat.

10. The method of claim 1, wherein the aldose reductase inhibitor is not a nitric oxide inducer.

11. The method of claim 1, wherein the aldose reductase inhibitor is administered at a dose of 0.01 to 1500 mg/day.

12. The method of claim 11, wherein the aldose reductase inhibitor is administered at a dose of 1 to 800 mg/day.
